(19) United States
(12) Patent Application Publication
(10) Pub. No.: US 2025/0195570 A1
(43) Pub. Date: Jun. 19, 2025

(54) METHODS AND COMPOSITIONS FOR CANCER THERAPY

(71) Applicant: THE UNIVERSITY OF CHICAGO, Chicago, IL (US)

(72) Inventors: Ralph WEICHSELBAUM, Chicago, IL (US); Hua LIANG, Chicago, IL (US); Chuan HE, Chicago, IL (US); Liangliang WANG, Chicago, IL (US)

(73) Assignee: THE UNIVERSITY OF CHICAGO, Chicago, IL (US)

(21) Appl. No.: 18/982,224

(22) Filed: Dec. 16, 2024

Related U.S. Application Data
(60) Provisional application No. 63/610,296, filed on Dec. 14, 2023.

(51) Int. Cl. A61K 35/15; A61K 9/00; A61P 35/00; C07K 16/28; C12N 5/078

(52) U.S. Cl. CPC A61K 35/15; A61K 9/0019; A61P 35/00; C07K 16/2818; C07K 16/2896; C12N 5/0634; C12N 2510/00

(57) ABSTRACT

Disclosed are compositions and methods useful for cancer treatment. The composition and methods include overexpressing BAMBI in a population of immune cells, such as myeloid-derived suppressor cells, in order to improve the immune cells immunogenic and/or anti-tumor effects. Also disclosed are compositions and methods for determining responsiveness to a radiotherapy and/or immunotherapy.

This application claims priority of U.S. Provisional Application No. 63/610,296 filed Dec. 14, 2023, which is hereby incorporated by reference in its entirety.

This invention was made with government support under CA195075 and HG008935 awarded by the National Institutes of Health. The government has certain rights in the invention.

### BACKGROUND OF THE INVENTION

### I. Field of the Invention

This invention relates to the field of cancer biology, oncology, radiology, immunology, and medicine.

### II. Background

Radiation therapy (RT) is a common treatment option for cancer patients, with an approximately 40% cure rate (1). Radiation simultaneously induces biological effects on cancer cells and elicits an immune response (2). However, the immunogenic effects of RT are counterbalanced by immune suppressive effects and thereby contribute to treatment failure. Immune suppressive mechanisms induced by RT include: recruitment of radioresistant suppressor cells (including Myeloid-derived-suppressor-cells MDSCs, Treg, etc) (3, 4); induction of soluble suppressive factors including programmed cell death-ligand 1 (PD-L1) and transforming growth factor β (TGF-β) (5, 6). These can serve as the factors of extrinsic radioresistance, highlighting an urgent need for other agents/therapies combine with RT to overcome extrinsic radioresistance and benefiting the patients.

MDSCs are immature myeloid-lineage cells and are specially associated with advanced cancer stage and adversely affect overall survival in cancer patients receiving RT (7, 8, 9). MDSCs suppress the antitumor immune response by secretion or expression of immunoregulatory factors (ARG1, nitric oxide, TGF-β, interleukin (IL)-10 and others) (10, 11). Among these, targeting TGF-β has shown mixed results in the preclinical and clinical settings owing to its complex pleiotropic function as both oncogenic and tumor-suppressive (12, 13, 14). Despite this, myeloid-specific TGF-β signaling acts as a critical mediator in tumor progression (15, 16, 17). Given that targeting TGF-β signaling in MDSCs may be an important therapeutic approach to enhance RT efficacy, it is essential to clarify the regulation of TGF-β signaling in MDSCs.

Bone morphogenetic protein and activin membrane-bound inhibitor (BAMBI) is a transmembrane glycoprotein that has a similar extracellular ligand binding domain structure as TGFβR1 but lacks an intracellular serine/threonine kinase domain (18). BAMBI negatively regulates TGF-β signaling, acting as a TGF-β pseudoreceptor (19, 20). Previous studies have established that BAMBI is controlled by SMAD3/4 signaling and Wnt/-βcatenin signaling in colon cancer cells (21, 22). DNA methylation is reported to be involved in BAMBI regulation in high-grade bladder cancer (23). These studies suggest that BAMBI expression is regulated both at the transcriptomic and epigenetic levels in cancer. Moreover, studies recently identified that the N⁶-methyladenosine (m⁶A) reader proteins and the epitranscriptome play a critical role in MDSC suppressive function after IR (24). However, role of epitranscriptional regulation and functional significance of BAMBI in immune cells is poorly understood.

Activation of transforming growth factor β (TGF-β) signaling serves as an extrinsic resistance mechanism that limits the potential for radiotherapy. Bone morphogenetic protein and activin membrane-bound inhibitor (BAMBI) antagonizes TGF-β signaling and is implicated in cancer progression. However, the molecular mechanisms of BAMBI regulation in immune cells and its impact on antitumor immunity after radiation have not been established.

### SUMMARY OF THE INVENTION

The disclosure generally relates to the discovery that introducing and/or overexpressing a bone morphogenetic protein and activin membrane-bound inhibitor (BAMBI) gene product in immune cells in a patient can improve the immunogenicity of the immune cells, can improve the patient's responsiveness to a cancer therapy, and can reduce tumor burden and/or aggressiveness in the patient.

Disclosed herein are populations of immune cells comprising an exogenous BAMBI gene product. In certain aspects, the population comprises myeloid-derived suppressor cells. The population of immune cells can include or exclude one or more different types of immune cells. In certain aspects, the population of immune cells comprises, consists of, or consist essentially of immature cells. In certain aspects, the population of immune cells comprises, consists of, or consist essentially of myeloid-derived suppressor cells. In certain aspects, the population of immune cells comprises, consists of, or consist essentially of myeloid precursor cells. The population of immune cells may be from any source, including from any patient disclosed herein.

The population of immune cells can include any exogenous BAMBI gene product, including a BAMBI RNA and/or a BAMBI protein. The exogenous BAMBI gene product can be introduced to the population of immune cells by any suitable means, including by a viral vector and/or a non-viral vector. Viral vectors may comprise an intact virus and/or compositions, such as one or more nucleic acids and/or proteins, capable of generating a virus. The viral vector can encode any BAMBI gene product described herein. In certain aspects, the viral vector comprises an adeno-associated virus, an adenovirus, a retrovirus, a lentivirus, a herpes simplex virus, or a combination thereof. It is also specifically contemplated that, in certain aspects, the exogenous BAMBI gene product is not introduced to the population of immune cells by a viral vector. It is also specifically contemplated that, in certain aspects, the viral vectors does not comprise an adeno-associated virus, an adenovirus, a retrovirus, a lentivirus, a herpes simplex virus, or a combination thereof. In certain aspects, the viral vector comprises an adeno-associated virus. In certain aspects, the non-viral vector comprises a lipid delivery system, a cationic polymer, a conjugate complex, or a combination thereof. It is also specifically contemplated that, in some aspects, the non-viral vector does not comprise a lipid delivery system, a cationic polymer, a conjugate complex, or a combination thereof. In certain aspects, the non-viral vector comprises naked DNA, naked RNA, a chromosome, a plasmid, or a combination thereof. It is also specifically contemplated that, in some aspects, the non-viral vector does not comprise naked DNA, naked RNA, a chromosome, a plasmid, or a combination thereof. It is also specifically contemplated that, in certain aspects, the exogenous BAMBI gene product is not introduced to the population of immune cells by a non-viral vector.

The exogenous BAMBI gene product can be expressed into BAMBI protein within the cells of the population of immune cells. The exogenous BAMBI gene product can cause an increase in BAMBI protein within individual cells of the population of immune cells. The exogenous BAMBI gene product can cause an increase in the level of BAMBI within the population of immune cells compared to a population of immune cells that do not comprise the BAMBI gene product, wherein the exogenous BAMBI gene product induces an overexpression of BAMBI protein in the population of immune cells. In certain aspects, the exogenous gene product induced overexpression in the population of immune cells. The overexpression may be relative to a population of immune cells that do not contain the exogenous BAMBI gene product.

In certain aspects, the population of immune cells does not comprise a specific type of immune cell, such as a B-cell, a T-cell, macrophages, NK cells, monocytes, neutrophils. In certain aspects, the population of immune cells does not comprise mature immune cells. In certain aspects, the population of immune cells does not comprise unmodified cells, which can include cells that have not been manipulated. Unmodified cells can include cells that have not been introduced exogenous materials, such as any exogenous gene product disclosed herein. Unmodified cells can include cells that have not been genetically engineered.

Also disclosed are pharmaceutical compositions comprising a population of immune cells, including any population of immune cells disclosed herein. The pharmaceutical composition can comprise, consist of, or consist essentially of any of the immune cells disclosed herein. The pharmaceutical composition can comprise a population of myeloid-derived suppressor cells overexpressing a BAMBI gene product. The pharmaceutical composition can comprise excipients suitable for delivery of the population of immune cells, BAMBI overexpression construct, and/or BAMBI gene product. In certain aspects, the pharmaceutical composition comprises a population of immune cells between approximately 1×10³ to 9×10¹² cells, including any of the cells disclosed herein.

Also disclosed are methods of enhancing a patient's responsiveness to a cancer therapy, methods of enhancing the effectiveness of a cancer therapy, methods of sensitizing a patient to a cancer therapy, methods of increasing an immune response against a cancer, methods of treating a cancer, methods of preventing a cancer, methods of decreasing cancer progression, and methods of improving a patient's prognosis. The methods can comprise 1, 2, 3, 4, 5, 6, 7 or more of any of the following steps: increasing expression of a BAMBI gene product, inducing overexpression of a BAMBI gene product in a population of immune cells, administering a BAMBI overexpression construct to a patient, administering a cancer therapy to a patient, administering a pharmaceutical composition to the patient, determining expression of BAMBI in the patient, measuring an amount of BAMBI in a population of immune cells, and monitoring the patient. It is contemplated that the preceding steps can occur in any order and each step may be performed one or more times, in any order relative to the other steps. For example, in certain aspects, the increasing expression step may occur before, during, and/or after the administering a cancer therapy step. It is also specifically contemplated, in certain aspects, that any of the preceding steps may be excluded from the methods. The BAMBI gene product may be increased and/or overexpressed in autologous cells and/or allogeneic cells relative to the patient. The increasing expression may occur in vivo and/or ex vivo. The inducing overexpression may occur in vivo and/or ex vivo. In certain aspects, the patient is a human. In certain aspects, the patient is a mammal.

In certain aspects, the cancer therapy comprises a therapy that modulates the immune system in the patient receiving the cancer therapy, such as radiotherapy and/or an immunotherapy. In certain aspects, the immunotherapy comprises a checkpoint blockade therapy. In certain aspects, the checkpoint blockade therapy includes or excludes an anti-PD-L1 molecule, an anti-PD-1 molecule, an anti-CTLA-4 molecule, and anti-LAG-3 molecule, or a combination thereof. Such molecules may be antibodies, small molecule inhibitors, or other molecules capable of blocking the function of the checkpoint molecule. In certain aspects, the checkpoint blockade therapy includes or excludes pembrolizumab, nivolumab, cemiplimab, atezolizumab, avelumab, durvalumab, ipilimumab, tremelimumab, relatlimab, or a combination thereof. In certain aspects, the radiotherapy includes or excludes ionizing radiation.

In certain aspects, the patient has been determined to have reduced expression of BAMBI. In certain aspects, the determining can comprise measuring expression of BAMBI in a sample from the patient. The sample can be any sample, including a sample comprising a population of immune cells. The population of immune cells can comprise, consist of, or consist essentially of myeloid-derived-suppressor-cells. In certain aspects, the reduced expression of BAMBI comprises a reduced expression of BAMBI in myeloid-derived-suppressor-cells in the patient. In certain aspects, reduced expression is relative to a standard. The standard can be determined by one skilled in the art and can be an empirically determined standard based on experimental evidence. In certain aspects, the standard comprises expression of BAMBI in a patient that has not received a radiotherapy. In certain aspects, the standard comprises expression of BAMBI in myeloid-derived-suppressor-cells that have not been exposed to a radiotherapy. In certain aspects, the standard comprises expression of BAMBI in a healthy individual or an average expression of BAMBI in a population of individuals. In certain aspects, the standard comprises expression of BAMBI in myeloid-derived-suppressor-cells in a healthy individual or an average expression of BAMBI in myeloid-derived-suppressor-cells in a population of individuals.

Disclosed are methods comprising increasing expression of bone morphogenetic protein and activin membrane-bound inhibitor (BAMBI) in a population of immune cells in a patient. The population of immune cells may comprise, consist of, or consist essentially of myeloid-derived suppressor cells. The population of immune cells may be any population of immune cells disclosed herein.

In certain aspects, increasing expression of BAMBI comprises administering a BAMBI overexpression construct, including any BAMBI expression construct disclosed herein (such as any population of immune cells disclosed herein and/or any viral vector and/or non-viral vector disclosed herein), to the patient. In certain aspects, the BAMBI overexpression construct comprises a viral vector encoding a BAMBI gene product, including any viral vector disclosed herein. In certain aspects, the viral vector comprises an adeno-associated virus, an adenovirus, a retrovirus, a lentivirus, a herpes simplex virus, or a combination thereof. In certain aspects, the BAMBI overexpression construct comprises a non-viral vector encoding a BAMBI gene product, including any non-viral vector disclosed herein. The non-viral vector can comprise a lipid delivery system, a cationic polymer, a conjugate complex, or a combination thereof. The non-viral vector can comprise naked DNA, naked RNA, a chromosome, a plasmid, or a combination thereof.

Also disclosed are methods comprising increasing expression of BAMBI, where increasing the expression comprises administering any population of immune cells disclosed herein and/or any pharmaceutical composition disclosed herein.

In certain aspects, administering comprises intratumoral administration, intravenous administration, intramuscular administration, subcutaneous administration, oral administration, parenteral administration, intraperitoneal administration, or a combination thereof.

In certain aspects, the patient has, is suspected of having, or has been diagnosed with having lung cancer, breast cancer, prostate cancer, skin cancer, colorectal cancer, kidney cancer, bladder cancer, a blood cancer, thyroid cancer, or endometrial cancer.

In certain aspects, the patient has received a cancer therapy (such as a radiotherapy and/or immunotherapy) at least, at most, or approximately 1, 2, 3, 4, 5, 6, 7, 8, 9, 10, or more (or any range derivable therein) hours, days, weeks, or months before increasing expression of BAMBI in a population of immune cells. In certain aspects, the patient has received a cancer therapy (such as a radiotherapy and/or immunotherapy) at least, at most, or approximately 1, 2, 3, 4, 5, 6, 7, 8, 9, 10, or more (or any range derivable therein) hours, days, weeks, or months after increasing expression of BAMBI in a population of immune cells. In certain aspects, the patient receives the cancer therapy concurrently with receiving a BAMBI overexpression construct. In certain aspects, the patient receives the cancer therapy while a BAMBI gene product is overexpressed in a population of immune cells in the patient. In certain aspects, the patient receives the cancer therapy concurrently with increasing the expression of a BAMBI gene product. In certain aspects, the patient has received the cancer therapy prior to the increasing expression of BAMBI, including 1, 2, 3, 4, 5, 6, 7, 8, 9, 10, or more (or any range derivable therein) hours, days, weeks, or months prior to the increasing. In some aspects, the patient receives the cancer therapy during the increasing expression of BAMBI. In certain aspects, the patient receives the cancer therapy after the increasing expression of BAMBI, including 1, 2, 3, 4, 5, 6, 7, 8, 9, 10, or more (or any range derivable therein) hours, days, weeks, or months after the increasing.

In certain aspects, the increasing expression is compared to an unmodified population of cells. In certain aspects, the increasing expression is relative to a population of immune cells that have not been manipulated. In certain aspects, the increasing expression is relative to a population of immune cells that have not been introduced any of the overexpression constructs disclosed herein. In certain aspects, the increasing expression is relative to a population of immune cells that do not have an exogenous BAMBI gene product.

Also disclosed are BAMBI overexpression constructs, including any of the viral vectors and/or non-viral vectors encoding a BAMBI gene product disclosed herein.

Also disclosed are methods of predicting responsiveness to a radiotherapy and/or immunotherapy in a patient, methods of predicting radiotherapy and/or immunotherapy response, methods of predicting cancer survival, methods of diagnosing a patient with radiotherapy and/or immunotherapy refractory cancer, methods of prognosing a patient, methods of determining a treatment plan for a patient. The methods can comprise 1, 2, 3, or more steps including of any of the following steps: measuring an amount of bone morphogenetic protein and activin membrane-bound inhibitor (BAMBI) in a population of immune cells, measuring an amount of bone morphogenetic protein and activin membrane-bound inhibitor (BAMBI) in a sample from the patient, comparing the amount of BAMBI to a standard. In certain aspects, one or more of the preceding steps are specifically excluded from the method.

In certain aspects, the patient has received, will receive, and/or is indicated to receive radiotherapy. In certain aspects, the patient has received, will receive, and/or is indicated to receive immunotherapy. In certain aspects, the patient has received, will receive, and/or is indicated to receive radiotherapy and immunotherapy. One skilled in the art can determine whether a patient is indicated to receive a radiotherapy. For example, patients indicated to receive radiotherapy include patient having a tumor that cannot be surgically removed. In certain aspects, the immunotherapy comprises a checkpoint blockade therapy. In certain aspects, the patient has, is suspected of having, or has been diagnosed with having lung cancer, breast cancer, prostate cancer, skin cancer, colorectal cancer, kidney cancer, bladder cancer, a blood cancer, thyroid cancer, or endometrial cancer. In certain aspects, the checkpoint blockade therapy comprises an anti-PD-L1 molecule, an anti-PD-1 molecule, an anti-CTLA-4 molecule, or a combination thereof. In certain aspects, the radiotherapy comprises ionizing radiation.

Disclosed are methods comprising comparing the amount of BAMBI to a standard. In certain aspects, the standard comprises expression of BAMBI in a patient that has not received a radiotherapy. In certain aspects, the standard comprises expression of BAMBI in myeloid-derived-suppressor-cells that have not been exposed to a radiotherapy. In certain aspects, the standard comprises expression of BAMBI in a healthy individual or an average expression of BAMBI in a population of individuals. In certain aspects, the standard comprises expression of BAMBI in myeloid-derived-suppressor-cells in a healthy individual or an average expression of BAMBI in myeloid-derived-suppressor-cells in a population of individuals.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” “(x and z) or y,” or “x or y or z.” It is specifically contemplated that x, y, or z may be specifically excluded from an aspect or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is contemplated that any aspect discussed in this specification can be implemented with respect to any method or composition of the invention, and vice versa. Furthermore, compositions of the invention can be used to achieve methods of the invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also aspects that may be implemented in the context of aspects discussed elsewhere in a different Example or elsewhere in the application, such as in the Summary of the Invention, Brief Description of the Drawings, Detailed Description of the Invention, and/or Claims.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1H: IR specifically reduces BAMBI expression in MDSCs in humans and murine cancers. (A) tSNE clustering of flow cytometry marker expression profiles in live CD45⁺ cells in PBMCs from metastatic NSCLC patients enrolled in a clinical trial (the COSINR study, NCT03223155). Expression intensity of CD33, CD14, HLA-DR and BAMBI. (B) Mean Fluorescent Intensity (MFI) of BAMBI in different immune cells in PBMCs as (A). mMDSC (CD33⁺CD14^{hi}HLA-DR^{low}). (C) Heatmap showing the changes of Bambi expression in different myeloid cell clusters in control (ctrl) and irradiated (IR) tumors respectively, based on the scRNA-seq data of CD45⁺ immune cells. CD45⁺ cells were obtained from four pooled MC38 tumor-bearing mice four days after IR. P values were calculated by a Wilcoxon-Mann-Whitney test (alternative=“greater”). (D-E) Mean Fluorescent Intensity (MFI) of BAMBI (D) and representative flow cytometry analysis (E) of BAMBI expression in MC38 tumor-infiltrating MDSCs three days after IR. (F) Relative mRNA levels of Bambi based on the RNA-seq analysis of CD11b⁺ myeloid cells isolated from non-irradiated or irradiated MC38 tumors, three days after IR (20 Gy). (G) qPCR analysis of Bambi in MDSCs (CD45⁺CD11b⁺Ly6C^{hi}) isolated from non-irradiated or irradiated MC38 tumors, three days after IR (20 Gy). (H) Flow cytometry analysis of BAMBI (MFI) in mMDSCs in PBMCs from metastatic NSCLC patients enrolled in a clinical trial (the COSINR study, NCT03223155) (pre-RT vs. post-RT). The blood samples were collected immediately post hypofractionated radiation, approximately 1-3 weeks (median 14 days) after the pre-RT samples. Data are represented as mean±s.e.m., n, number of mice. One of two or three representative experiments was shown. Statistical analysis was performed using two-sided unpaired Student's t-test (C, E, and G). *P<0.05 and **P<0.01.

FIGS. 2A-2F: IR-YTHDF2 axis regulates BAMBI expression in MDCSs in an m⁶A-dependent manner (A) Integrative Genomics Viewer tracks displaying the distribution of m⁶A peaks and YTHDF2-binding peaks across the Bambi transcripts, based on the MeRIP-seq and RIP-seq data of MC38 tumor-infiltrating myeloid cells. (B) Graphs showing enrichment of Bambi mRNA in the YTHDF2-immunoprecipitated RNA fraction of bone marrow-derived MDSCs, determined by RIP-qPCR (right bar). Rabbit IgG served as a control (left bar). Enrichment of the indicated genes was normalized to the input level. (C) MDSCs were sorted from bone marrow-derived cells from WT and Ythdf2-cKO (Lyz2^{cre}Ythdf2^{f/f}) mice; WT-MDSCs were directly treated with IR (4 Gy and cultured for 6 hr) (WT+IR). MDSCs were treated with actinomycin D. mRNA was collected at indicated time points after treatment and mRNA level of Bambi was measured by RT-qPCR. (D) qPCR analysis of Bambi mRNA level in different MDSCs isolated from MC38 tumors in WT, WT+IR, Ythdf2-cKO, Ythdf2-cKO+IR mice, three days after IR. (n=3 per group) (E) Mean Fluorescent Intensity (MFI) of BAMBI in MC38 tumor infiltrating MDSCs (as indicated) by flow cytometry analysis, three days after IR. (F) The WT-YTHDF2 and m⁶A-binding-site-mutated YTHDF2-overexpressing Ythdf2-deficient CD45.2-BM-MDSCs (Ythdf2-cKO+WT, Ythdf2-cKO+Mut respectively) were obtained via lentiviral transfection. The BM-MDSCs were used for adoptive transfer into MC38 tumor-bearing CD45.1 mice. On the same day, the mice were treated with local tumor irradiation (one dose of 20 Gy). Three days after IR, tumors were harvested to measure the MFI of BAMBI level in newly infiltrated CD45.2-MDSCs by flow cytometry. Data are represented as mean±s.e.m., n, number of mice. One of two or three representative experiments was shown. Statistical analysis was performed using two-sided unpaired Student's t-test (B), or one-way ANOVA with Bonferroni's multiple comparison tests (C-F). *P<0.05, **P<0.01, ***P<0.001, and ****P<0.0001.

FIGS. 3A-3G: BAMBI suppresses MDSC migration and suppressive function via inhibiting TGF-β signaling. (A) WT and Bambi knockdown BM-MDSCs as indicated were used for the transwell assay. The attached cells on the transwell membrane were visualized under a light microscope and quantified. (n=3 per group) (B) MC38 tumor bearing Ccr2-knockout mice (CD45.2) were adoptively transferred with 1×10⁶ WT, Bambi-knockdown (Bambi-KD), BAMBI-overexpressing (BAMBI-OE) BM-MDSCs (CD45.1) via i.v. injection. On the same day, mice were treated with local IR (20 Gy, one dose). Three days after IR, the number of tumor-infiltrating CD45.1⁺CD11b⁺Ly6C^{hi} cells was determined by flow. (n=4 per group) (C) Heatmap showing the qPCR analysis of relative Bambi, Ccr2, Ccl24, Cx3crl, Mmp14, Cxcl10, Ccr5, and Il10 mRNA expression in WT, Bambi-KD, BAMBI-OE (BAMBI-overexpression) MDSCs. The qPCR data were normalized to Gapdh. (D) Flow cytometry analysis of an in vitro proliferation assay showing the frequency of proliferating CD8⁺ T cells when co-cultured with WT, Bambi-KD, and BAMBI-OE MDSCs. (E) Immunoblot analysis of signaling associated proteins and phosphorylated (p-) proteins (as indicated) in WT, Bambi-KD, and BAMBI-OE DSCs treated with TGF-β. (F) WT, Bambi-KD, Tgfbr2-KO, and Bambi-KD-Tgfbr2-KO BM-MDSCs were used for adoptive transfer into MC38 tumor bearing Ccr2-knockout mice. On the same day, mice were treated by with tumor-local IR (20 Gy, one dose). Three days after IR, the number of tumor-infiltrating CCR2⁺CD11b⁺Ly6C^{hi} cells was determined by flow. (n=4 per group) (G) Flow cytometry analysis of an in vitro proliferation assay showing the frequency of proliferating CD8⁺ T cells when co-cultured with WT, Bambi-KD, Tgfbr2-KO, and Bambi-KD-Tgfbr2-KO BM-MDSCs. Data are represented as mean±s.e.m., n, number of mice. One of two or three representative experiments was shown. Statistical analysis was performed using two-sided unpaired Student's t-test (A, F, and G) or one-way ANOVA with Bonferroni's multiple comparison tests (B, D). *P<0.05, **P<0.01, ***P<0.001, and ****P<0.0001.

FIGS. 4A-4F: Enhancing Bambi improves the response to radiotherapy and immunotherapy. (A) CD11b-DTR bone marrow chimeric mice were injected subcutaneously with 1×10⁶ MC38 cells. When the tumor size reached 100 mm³, mice were injected with Diphtheria toxin (50 ng/per mice). Next day, the mice were adaptive transferred with BM-MDSCs (CD45.1) as indicated and were treated with tumor-local IR (20 Gy, one dose) on the same day. Tumor growth was monitored. (B-C) MC38 tumor-bearing mice were treated with tumor-local IR (20 Gy, one dose). On the same day, the mice were introtumoral injected with 2×10¹⁰ v.p. of AAV-Null (ctrl), AAV-Bambi (twice weekly, three doses totally). Tumor growth (B) and animal survival (C) was monitored. (D) Lyz2^{cre}TeCsf1r-DTR mice were injected subcutaneously with 1×10⁶ MC38 cells. When the tumor size reached 100 mm³, mice were injected with Diphtheria toxin (50 ng/per mice). Next day, the mice were treated with tumor-local IR (20 Gy, one dose) or AAV-Bambi (i.t. of with 2×10¹⁰ v.p.) as indicated. Tumor growth was monitored. (E) Lung metastasis in LLC tumor-bearing WT mice with indicated different treatment (22 days after IR). The size of lung metastases was measured. (n=10 per group) (F) WT mice were injected subcutaneously with 1×10⁶ B16F1 cells. When the tumor size reached 100 mm³, tumors were treated with anti-PD-L1 antibody (2 doses per week, three doses total), AAV-Bambi (twice weekly, three doses totally) and/or local IR (20 Gy, one dose), as indicated. Tumor growth was monitored. Data are represented as mean±s.e.m., n, number of mice. Statistical analysis was performed using two-way ANOVA test with corrections for multiple variables (A, B, D, and F), two-sided log-rank (Mantel-Cox) test (C). *P<0.05, **P<0.01, ***P<0.001, and ****P<0.0001.

FIGS. 5A-5I: BAMBI expression correlates with patient's outcome and immune responses. (A) Overall survival analysis of cancer patients in PanCancer, kidney renal clear cell carcinoma (KIRC), kidney renal papillary cell carcinoma (KIRP), phcochromocytoma and paraganglioma (PCPG), and uterine corpus endometrial carcinoma cohorts (UCEC) using the median value as a cutoff. High-BAMBI (expression≥median value); Low-BAMBI (expression<median value). Normalized gene expression and corresponding clinical data on patients were obtained from KM-plotter. (B-C) Correlation of BAMBI expression and infiltration level of CD8⁺ T cells (B) and MDSCs (C) in Bladder Carcinoma (BLCA) and KIRC cohorts, obtained by TIMER2.0. R represents Spearman's coefficient. (D) Relative mRNA level of Bambi in WT (left bar) and BAMBI-overexpressing (BAMBI-OE; right bar) MC38 (left graph) and B16F1 (right graph) cells. (n=3 per group). (E) Immunoblot analysis of BAMBI in WT and BAMBI-OE MC38 or BAMBI-OE B16F1 cells (Left). Immunoblot analysis of BAMBI in WT (sh-ctrl) and BAMBI-KD (sh-Bambi) MC38 or B16F1 cells (Right). (F-G) WT C57BL/6 mice were injected subcutaneously with 1×10⁶ MC38 or BAMBI-OE MC38 cells (F) and B16F1 or BAMBI-OE B16F1 cells (G). Tumor growth was monitored. (n=5 mice per group) (H-I) WT C57BL/6 mice were injected subcutaneously with 1×10⁶ MC38 (sh-ctrl) or BAMBI-KD (sh-Bambi) MC38 cells (H) and B16F1 (sh-ctrl) or BAMBI-KD (sh-Bambi) B16F1 cells (I). Tumor growth was monitored. (n=5 mice per group) Data are represented as mean±s.e.m. One of two or three representative experiments was shown (D-I). Statistical analysis was performed using two-sided unpaired Student's t-test (D) or two-way ANOVA test with corrections for multiple variables (F-I). *P<0.05, **P<0.01, and ***P<0.001.

FIGS. 6A-6D: Distribution of BAMBI expression in different immune cells in both humans and murine cancers. (A) Heatmap showing the Mean Fluorescent Intensity (MFI) of BAMBI in different MC38 tumor-infiltrating immune cells, including CD4⁺ T cells (CD45⁺CD4⁺), Treg cells (CD45⁺CD4⁺ CD25⁺), CD8⁺ T cells (CD45⁺CD8⁺), DCs (CD45⁺CD11c⁺MHCII⁺), Macrophages (CD45⁺CD11b⁺F4/80⁺), mMDSCs (CD45⁺CD11b⁺Ly6C^{hi}Ly6G⁻), PMN-MDSCs (CD45⁺CD11b⁺Ly6C⁻Ly6G^{hi}), B cells (CD45⁺CD3⁻ CD19⁺), NK cells (CD45⁺ CD3⁻NK1.1⁺). (B) Violin plot showing the Bambi expression levels in different immune cells based on the public scRNA-seq data of murine colon cancer (GSE122969), obtained by TISCH2. (C) Heatmap showing the BAMBI expression levels in different immune cells based on the public scRNA-seq data of SKCM and CRC patients, obtained by TISCH2. (D) tSNE clustering of flow cytometry marker expression profiles in live CD45⁺ cells in PBMCs from metastatic NSCLC patients enrolled in a clinical trial (the COSINR study, NCT03223155). Expression intensity of CD3, CD4, CD8 and CD56.

FIGS. 7A-7C: IR specifically reduces BAMBI expression in MDSCs. (A) Heatmap showing the Mean Fluorescent Intensity (MFI) of BAMBI in different MC38 tumor-infiltrating immune cells, (ctrl vs. IR, three days after IR) including CD4⁺ T cells (CD45⁺CD4⁺), Treg cells (CD45⁺CD4⁺ CD25⁺), CD8⁺ T cells (CD45⁺CD8⁺), DCs (CD45⁺CD11c⁺MHCII⁺), Macrophages (CD45⁺CD11b⁺F4/80⁺), mMDSCs (CD45⁺CD11b⁺Ly6C^{hi}Ly6G⁻), PMN-MDSCs (CD45⁺CD11b⁺Ly6C⁻Ly6G^{hi}), B cells (CD45⁺CD3⁻ CD19⁺), NK cells (CD45⁺ CD3⁻NK1.1⁺). (B) Mean Fluorescent Intensity (MFI) of BAMBI in CD45⁻ cells in nonirradiated (ctrl) vs. irradiated (IR) MC38 tumors (three days after IR). (C) The BAMBI mRNA levels in different cancer patients with lung/liver metastasis, based on the RNA-seq of whole tumor biopsy (pre-RT vs. post-RT, the COSINR study, NCT03223155). Data are represented as mean±s.e.m., n, number of mice Statistical analysis was performed using two-sided unpaired Student's t-test (B).

FIGS. 8A-8D: YTHDF2 down-regulates BAMBI expression in MDCSs. (A) Mean Fluorescent Intensity (MFI) of BAMBI in MDSCs in nonirradiated (ctrl, left bar) vs. irradiated (IR, right bar) MC38 tumors (three days after IR) from WT mice and Lyz2^{cre}Rela^{f/f} mice. (n=5 per group). (B) qPCR analysis of Bambi in MDSCs (CD45⁺CD11b⁺Ly6C^{hi}) isolated from nonirradiated (ctrl, left bar) vs. irradiated (IR, one dose of 20Gy, right bar) MC38 tumors (three days after IR) from WT mice and Lyz2^{cre}Rela^{f/f} mice. (n=5 per group). (C) The WT-YTHDF2 and m⁶A-binding-site-mutated YTHDF2-overexpressing Ythdf2-deficient CD45.2-BM-MDSCs (Ythdf2-cKO+WT, Ythdf2-cKO+Mut respectively) were obtained via lentivirus transfection. The BM-MDSCs were used for adoptive transfer into MC38 tumor-bearing CD45.1 mice. On the same day, the mice were treated with tumor local irradiation (one dose of 20 Gy). Three days after IR, tumors were harvested to measure the mRNA level of Bambi (by qPCR analysis) in newly infiltrated CD45.2-MDSCs by flow cytometry. (n=4 per group). (D) The Graphs showing enrichment of Bambi mRNA in the m⁶A-immunoprecipitated RNA fraction of BM-MDSCs, determined by RIP-qPCR. Rabbit IgG served as a control. Enrichment of the indicated genes was normalized to the input level. (n=5 per group). Data are represented as mean±s.e.m. One of two or three representative experiments was shown. Statistical analysis was performed using one-way ANOVA with Bonferroni's multiple comparison tests (A-C) or two-sided unpaired Student's t-test (D). *P<0.05, **P<0.01, ***P<0.001 and ****P<0.0001.

FIGS. 9A-9C: TGF-β signaling in myeloid cells alters the response to radiotherapy. (A) Heatmap showing the qPCR analysis of relative Ccr2, Ccl24, Cx3crl, Mmp14, and Cxcl10 mRNA expression in WT, Bambi-KD, Ythdf2-cKO (Lyz2^{cre}Ythdf2^{f/f}), and Bambi-KD-Ythdf2-cKO BM-MDSCs stimulated with rTGF-beta. (B) Heatmap showing the qPCR analysis of relative Ccr2, Ccl24, Cx3crl, Mmp 14, and Cxcl10 mRNA expression in WT, Bambi-KD, Tgfbr2-KO, and Bambi-KD-Tgfbr2-KO BM-MDSCs stimulated with rTGF-beta. (C) Wild-type (Tgfbr2^{fl/fl}) or Lyz^{cre}Tgfbr2^{fl/fl} mice were injected subcutaneously with 1×10⁶ MC38 cells. When the tumor size reached 100 mm³, tumor-bearing mice were treated with tumor-local IR (20 Gy, one dose). Tumor growth was monitored. Data are represented as mean±s.e.m., n, number of mice. One of two or three representative experiments was shown. Statistical analysis was performed using two-way ANOVA test with corrections for multiple variables (C). *P<0.05, **P<0.01, ***P<0.001 and ****P<0.0001.

FIGS. 10A-10F: Combination AAV-Bambi and IR treatment enhances immune responses. (A) The number of tumor-infiltrating CD45⁺CD11b⁺Ly6C^{hi} cells in mice transferred with indicated MDSCs, three days after IR, as assessed by flow cytometry. (B) Representative flow cytometry analysis of PE-(flag) BAMBI levels in tumor-infiltrating MDSCs (CD45⁺CD11b⁺Ly6C^{hi}Ly6G⁻), 24 h after AAV-Bambi (AAV9-CMV-Flag-Bambi) treatment (i.t. of 2×10¹⁰ v.p.). (C-D) The numbers of tumor-infiltrating CD45⁺CD8⁺ (C) and CD45⁺CD8⁺IFNγ⁺ (D) T cells in MC38 tumor-bearing mice with treatments as indicated seven days after IR. (n=5 per group); left bar=ctrl, left middle bar=IR, right middle bar=AAV-Bambi, right bar=AAV-Bambi+IR (E) MDSCs were isolated from MC38 tumor bearing mice and cocultured with naïve CD8⁺ T cells for three days. The concentration of IFN-γ was measured by CBA flex set. (F) LLC tumor-bearing mice were treated with tumor-local IR (20 Gy, one dose). On the same day, the mice were intratumorally injected with 2×10¹⁰ v.p. of AAV-Null (ctrl), AAV-Bambi (twice weekly, three doses totally). The primary tumor growth was monitored. Data are represented as mean±s.e.m., n, number of mice. One of two or three representative experiments was shown. Statistical analysis was performed using two-way ANOVA test with corrections for multiple variables (F), two-sided unpaired Student's t-test (E) or one-way ANOVA with Bonferroni's multiple comparison tests (A, C, D). *P<0.05, and **P<0.01.

FIG. 11: Schematic diagram of aspects disclosed herein.

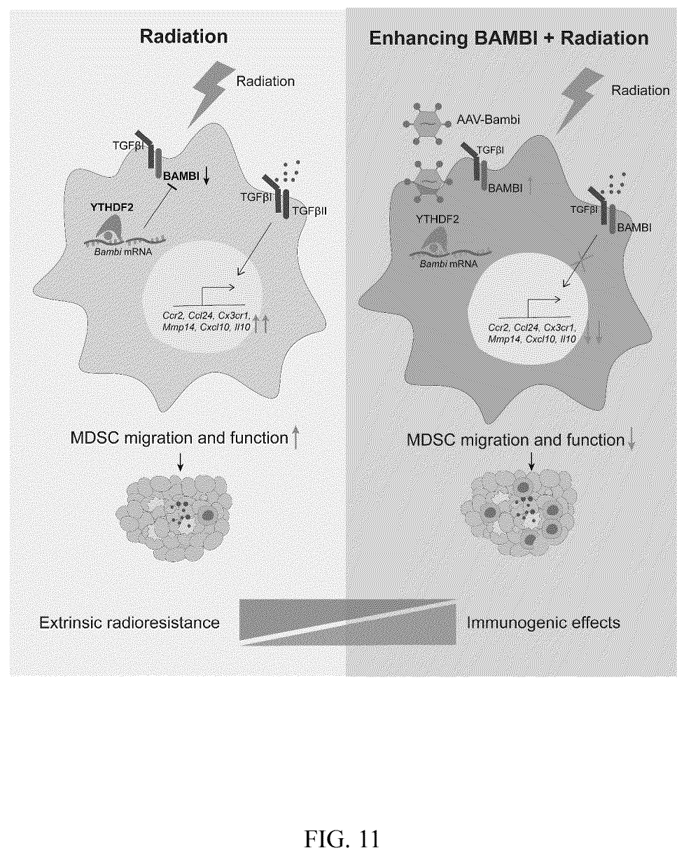

### DETAILED DESCRIPTION OF THE INVENTION

### I. General Aspects of the Disclosure

Aspects herein demonstrate that ionizing radiation (IR) specifically reduces BAMBI expression, including in immunosuppressive myeloid-derived-suppressor-cell (MDSC). In certain aspects, such reduction occurs in murine models. In certain aspects, such reduction occurs in humans. In some aspects, YTHDF2 directly binds and degrades Bambi transcripts in an m⁶A-dependent manner and this relies on NF-κB signaling. In some aspects, BAMBI suppresses the tumor infiltrating capacity and suppression function of MDSCs via inhibiting TGF-β signaling. In certain aspects, adeno-associated viral delivery of Bambi (AAV-Bambi) to the tumor microenvironment boosts the antitumor effects of radiotherapy and radio-immunotherapy combinations. In certain aspects, combination with AAV-Bambi and IR not only improves local tumor control but also suppresses distant metastasis, further supporting its clinical translation potential. Certain aspects herein uncover a surprising role of BAMBI in myeloid cells, unveiling a therapeutic strategy that may, in some aspects, overcome extrinsic radioresistance.

In certain aspects, IR reduces BAMBI expression in human and/or mouse MDSC, such as by NF-κB-mediated induction of YTHDF2. In some aspects, YTHDF2 recognition of methylated Bambi transcripts signals them for degradation in an m⁶A-dependent manner. In certain aspects, either enhancing BAMBI expression in myeloid cells and/or in situ delivery of BAMBI via adeno-associated virus (AAV-Bambi) augmented the antitumor effects of radiotherapy and radio-immunotherapy combinations.

### II. Administration of Therapeutic Compositions

The therapy provided herein may comprise administration of a combination of therapeutic agents, such as a first cancer therapy (e.g., a radiotherapy or an immunotherapy, for example, a checkpoint inhibitor therapy) and a composition capable of increasing BAMBI expression in a population of immune cells (a “BAMBI composition”). The BAMBI composition may comprise any BAMBI overexpression construct described herein, such as any viral vector and/or non-viral vector disclosed herein. The BAMBI composition may comprise any population of cells disclosed herein, including any population of cells comprising an exogenous BAMBI gene product. The therapies may be administered in any suitable manner known in the art. For example, the BAMBI composition and the cancer therapy may be administered sequentially (at different times) or concurrently (at the same time or approximately the same time; also “simultaneously” or “substantially simultaneously”). In some aspects, the BAMBI composition and the cancer therapy are administered in separate compositions. In some aspects, the BAMBI composition and the cancer therapy are in the same composition.

In some aspects, the BAMBI composition and the cancer therapy are administered substantially simultaneously. In some aspects, the BAMBI composition and the cancer therapy are administered sequentially. In some aspects, the BAMBI composition is administered before administering the cancer therapy. In some aspects, the BAMBI composition is administered after administering the cancer therapy. In some aspects, a first dose of the BAMBI composition is administered before administering the cancer therapy and further dose(s) of the BAMBI composition are administered after administering the cancer therapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intratumorally, intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a single dose of the BAMBI composition is administered. In some aspects, multiple doses of the BAMBI composition are administered. In some aspects, the BAMBI composition is administered at a dose of between 1 mg/kg and 5000 mg/kg. In some aspects, the BAMBI composition is administered at a dose of between 1 mg/kg and 5000 mg/kg. In some aspects, the BAMBI composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

In some aspects, the BAMBI composition is administered at a dose of between 1×10³ to 9×10¹² cells (or any range derivable therein), including when the BAMBI composition comprises cells disclosed herein, such as a population of immune cells disclosed herein. In some aspects, the BAMBI composition is administered at a dose of approximately 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹² cells (or any range derivable therein).

In some aspects, a single dose of the immunotherapy is administered. In some aspects, multiple doses of the immunotherapy are administered. In some aspects, the immunotherapy is administered at a dose of between 1 mg/kg and 100 mg/kg. In some aspects, the immunotherapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

In some aspects, the radiotherapy administered to the subject provides irradiation in a dose range of 0.5 Gy to 60 Gy. In some aspects, the radiotherapy administered to the subject provides irradiation at a dose of at least, at most, or about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 Gy. In some aspects, the radiotherapy is administered in a single dose. In some aspects, the radiotherapy is administered in a fractionated dose over a period of time of not more than one week. In some aspects, the radiotherapy is delivered in a fractionated dose over a period of time of not more than three days.

The quantity to be administered, both according to number of treatments and unit dose, depends on the treatment effect desired. The term “therapeutic benefit” or “therapeutically effective” as used throughout this application refers to anything that promotes or enhances the well-being of the subject with respect to the medical treatment of cancer. This includes, but is not limited to, a reduction in the frequency or severity of the signs or symptoms of a disease. For example, treatment of cancer may include but is not limited to total or partial remission of the cancer. Treatment of cancer may also refer to prolonging survival of a subject with a cancer. The term “therapeutically effective amount” refers to an amount sufficient to produce a desired therapeutic result, for example an amount of a BAMBI composition and/or a cancer therapy or a composition comprising such a BAMBI composition and/or a cancer therapy sufficient to improve at least one symptom of a medical condition in a subject to whom the BAMBI composition and/or a cancer therapy or composition thereof are administered.

In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### A. Cancer Therapy

In some aspects, the disclosed methods comprise administering a cancer therapy to a subject or patient. The cancer therapy may be chosen based on expression level measurements, alone or in combination with a clinical risk score calculated for the subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a systemic cancer therapy. In some aspects, the cancer therapy comprises an immunotherapy, which may be an immune blockade or immune checkpoint inhibitor therapy. In some aspects, the cancer therapy comprises radiotherapy, which can be ionizing radiation. Any of these cancer therapies may also be excluded. Combinations of these therapies may also be administered. For example, a cancer therapy may comprise a combination of an immunotherapy and radiotherapy.

Also disclosed are methods comprising measuring the level of certain gene products in a cancer patient and/or measuring the level of certain gene products in a subject having, suspected of having, or diagnosed with having cancer.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; amcloblastic odontosarcoma; ameloblastoma, malignant; ameloblastic fibrosarcoma; pincaloma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; cosinophilic leukemia; monocytic leukemia; mast cell leukemia; megakaryoblastic leukemia; myeloid sarcoma; and hairy cell leukemia.

### III. Obtaining Gene Products and Overexpression Constructs

In some aspects, there are nucleic acid molecules encoding a BAMBI gene product such as a BAMBI RNA and BAMBI protein. These may be generated by methods known in the art and expressed in any suitable recombinant expression system.

### A. Vectors

In some aspects, contemplated are expression vectors comprising a nucleic acid molecule encoding a polypeptide of the desired sequence, such as a BAMBI gene product, or a portion thereof. Expression vectors comprising the nucleic acid molecules may encode the BAMBI gene product. In some aspects, expression vectors comprising nucleic acid molecules may encode fusion proteins thereof. In addition to control sequences that govern transcription and translation, vectors and expression vectors may contain nucleic acid sequences that serve other functions as well.

To express the gene products, DNAs encoding the gene products can be inserted into expression vectors such that the gene area is operatively linked to transcriptional and translational control sequences. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use with an aspect to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 1. Host Cells

In another aspect, contemplated are the use of host cells into which a recombinant expression vector has been introduced. An expression construct encoding a BAMBI gene product can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### D. Viral Vectors

Disclosed herein are viral vectors encoding for a BAMBI gene product. The viral vectors may be capable of increasing expression of the BAMBI gene product. The viral vectors may be specifically engineered to be capable of increasing expression of the BAMBI gene product in a specific population of cells, such as any immune cell disclosed herein.

### 1. Adenoviral Infection

One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the some starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells.

### 2. Retroviral Infection

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

In some aspects, a retroviral vector of the present disclosure is a lentiviral vector.

### 3. AAV Infection

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present methods and compositions. Details concerning the generation and use of rAAV vectors are described in U.S. Pat. Nos. 5,139,941 and 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

### E. Non-Viral Delivery

In addition to viral delivery of the expression vectors, including those encoding a BAMBI gene product, the following are additional methods of recombinant gene delivery to a given host cell.

### 1. Lipid Mediated Transfection

In a further aspect of the invention, an expression vector may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Advances in lipid formulations have improved the efficiency of gene transfer in vivo (Smyth-Templeton et al., 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP: cholesterol lipid formulation is said to form a unique structure termed a “sandwich liposome”. This formulation is reported to “sandwich” DNA between an invaginated bi-layer or ‘vase’ structure. Beneficial characteristics of these lipid structures include a positive colloidal stabilization by cholesterol, two dimensional DNA packing and increased serum stability.

In further aspects, the liposome is further defined as a nanoparticle. A “nanoparticle” is defined herein to refer to a submicron particle. The submicron particle can be of any size. For example, the nanoparticle may have a diameter of from about 0.1, 1, 10, 100, 300, 500, 700, 1000 nanometers or greater. The nanoparticles that are administered to a subject may be of more than one size.

Any method known to those of ordinary skill in the art can be used to produce nanoparticles. In some aspects, the nanoparticles are extruded during the production process. Exemplary information pertaining to the production of nanoparticles can be found in U.S. Patent App. Pub. No. 20050143336, U.S. Patent App. Pub. No. 20030223938, and U.S. Patent App. Pub. No. 20030147966, each of which is herein specifically incorporated by reference into this section.

One of ordinary skill in the art would be familiar with use of liposomes or lipid formulation to entrap nucleic acid sequences. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

### IV. Isolation of Cells

Disclosed herein are populations of immune cells which can be isolated, purified, and/or modified from a source, such as a patient or other individual. The immune cells may be purified from a biological sample taken from the patient or other individual. The biological sample can be a whole blood sample. The biological sample can be a peripheral blood sample. In some aspects, immune cells are autologous. However the cells can be allogeneic. In some aspects, the immune cells are isolated from the patient, so that the cells are autologous. If the immune cells are allogeneic, the immune cells can be pooled from several donors. In certain aspects, the cells are administered to the subject of interest in an amount sufficient to control, reduce, or eliminate symptoms and signs of the disease being treated.

In some aspects, the immune cells are derived from the blood, bone marrow, lymph, umbilical cord, or lymphoid organs. In some aspects, the cells are human cells. The cells can be primary cells, such as those isolated directly from a subject and/or isolated from a subject and frozen. In some aspects, the methods include isolating cells from the subject, preparing, processing, culturing, and/or engineering them, as described herein, and re-introducing them into the same patient, before or after cryopreservation.

### V. Formulations and Culture of the Cells

In particular aspects, the cells of the disclosure, such as myeloid-derived suppressor cells and/or myeloid precursors, may be specifically formulated and/or they may be cultured in a particular medium. The cells may be cultured during the induction of BAMBI overexpression, including any BAMBI overexpression disclosed herein. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain aspects, the medium may comprise one, two, three, four, five, six, seven, eight, nine, ten, 11, 12, 13, 14, 15, 16, 17, 18, 19, 20 or more of the following: vitamins such as biotin; DL Alpha Tocopherol Acetate; DL Alpha-Tocopherol; vitamin A (acetate); proteins such as BSA (bovine serum albumin) or human albumin, fatty acid free Fraction V; catalase; human recombinant insulin; human transferrin; superoxide dismutase; other components such as Corticosterone; D-galactose; ethanolamine HCI; glutathione (reduced); L-Carnitine HCl; linoleic acid; linolenic acid; progesterone; putrescine 2HCl; sodium selenite; and/or T3 (triodo-I-thyronine). In specific aspects, one or more of these may be explicitly excluded.

In some aspects, the medium further comprises vitamins. In some aspects, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some aspects, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some aspects, the vitamins include or consist essentially of biotin, DL alpha tocopherol acetate, DL alpha-tocopherol, vitamin A, or combinations or salts thereof. In some aspects, the medium further comprises proteins. In some aspects, the proteins comprise albumin or bovine serum albumin, a fraction of BSA, catalase, insulin, transferrin, superoxide dismutase, or combinations thereof. In some aspects, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some aspects, the medium comprises one or more of the following: a B-27® supplement, xeno-free B-27® supplement, GS21™ supplement, or combinations thereof. In some aspects, the medium comprises or further comprises amino acids, monosaccharides, inorganic ions. In some aspects, the amino acids comprise arginine, cystine, isoleucine, leucine, lysine, methionine, glutamine, phenylalanine, threonine, tryptophan, histidine, tyrosine, or valine, or combinations thereof. In some aspects, the inorganic ions comprise sodium, potassium, calcium, magnesium, nitrogen, or phosphorus, or combinations or salts thereof. In some aspects, the medium further comprises one or more of the following: molybdenum, vanadium, iron, zinc, selenium, copper, or manganese, or combinations thereof. In certain aspects, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific aspects, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific aspects, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific aspects, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular aspects the cells are in a formulated cell suspension that is stable at room temperature for 1, 2, 3, or 4 hours or more from time of thawing.

### VI. Sample Preparation and Analysis

In certain aspects, methods involve obtaining a sample from a subject. The sample may be obtained in order to obtain a population of immune cells. The sample may comprise a population of immune cells that are, in various aspects, measured for expression of BAMBI. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain aspects the sample is obtained from a biopsy from esophageal tissue by any of the biopsy methods previously mentioned. In other aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the check, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### A. Nucleic Acid Assays

Various aspects include methods comprising measuring and/or assaying expression of BAMBI nucleic acid from a sample. The sample may comprise immune cells, such as myeloid-derived-suppressor-cells. The sample can be prepared for nucleic acid analysis, including using methods known in the art for isolating, purifying, measuring, and/or assaying nucleic acids. In some aspects, methods involve amplifying and/or sequencing one or more target genomic regions using at least one pair of primers specific to the target genomic regions. In certain aspects, the primers are 7, 8, 9, 10, 11, 12, 13, 14, 15, 16, 17, 18, 19, 20, 21, 22, 23, 24, or more (or any range derivable therein) nucleotides. In other aspects, enzymes are added such as primases or primase/polymerase combination enzyme to the amplification step to synthesize primers.

In some aspects, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040, 193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708, 153, 6,040, 193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, dd PCR (digital droplet PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form, although the single-stranded form is preferred.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one aspect, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one aspect, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one aspect, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

### B. Protein Assays

In some aspects, protein, including a BAMBI protein, may be analyzed by a protein assay. In some aspects, the protein assay comprises mass spectrometry, Western blotting, immunohistochemistry or other protein assay suitable for determining an expression level of BAMBI in a population of cells. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some aspects, protein is incubated with the endopeptidase, LysC and/or trypsin. The protein may be incubated with one or more protein cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some aspects, the cleaved proteins may be purified, such as by column purification. In certain aspects, purified peptides may be snap-frozen and/or dried, such as dried under vacuum. In some aspects, the purified peptides may be fractionated, such as by reverse phase chromatography or basic reverse phase chromatography. Fractions may be combined for practice of the methods of the disclosure. In some aspects, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some aspects, the raw mass spectrometry data may be processed and normalized using at least one relevant bioinformatics tool.

### EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1: IR Specifically Reduces BAMBI Expression in MDSCs in Humans and Murine Cancers

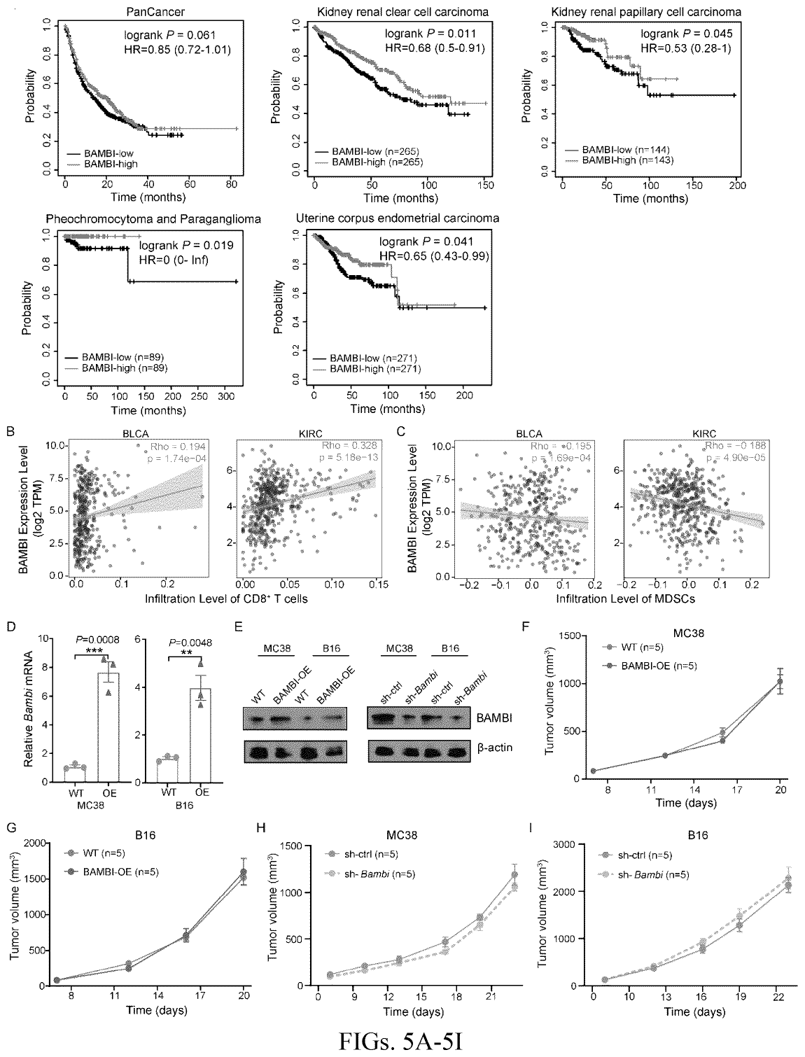

The inventors analyzed transcriptomic datasets of cancer patients to examine the potential relationship of BAMBI with cancer patient survival by leveraging the Kaplan Meier plotter website (25). Higher BAMBI expression was associated with prolonged overall survival in the PanCancer cohort (P=0.0077; FIG. 5A). The inventors observed a consistent correlation in six tumor types, including bladder carcinoma (BLCA; P=0.017) and kidney renal papillary cell carcinoma (KIRC; P=0.00052) (FIG. 5A). Furthermore, the exploratory database analysis using TIMER2.0 (Li et al., 2020) revealed that BAMBI was positively correlated with the CD8⁺ T cell tumor infiltration and negatively correlated with MDSC tumor infiltration in both BLCA and KIRC (FIGS. 5B-5C). Together, the in silico observations suggest that elevated BAMBI levels correlate with better survival and antitumor immunity among cancer patients.

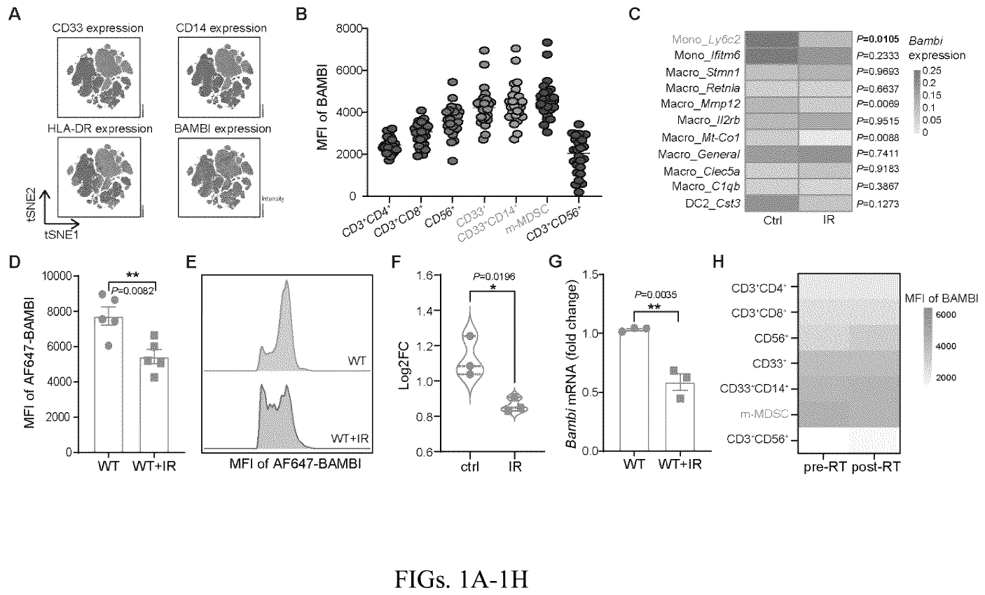

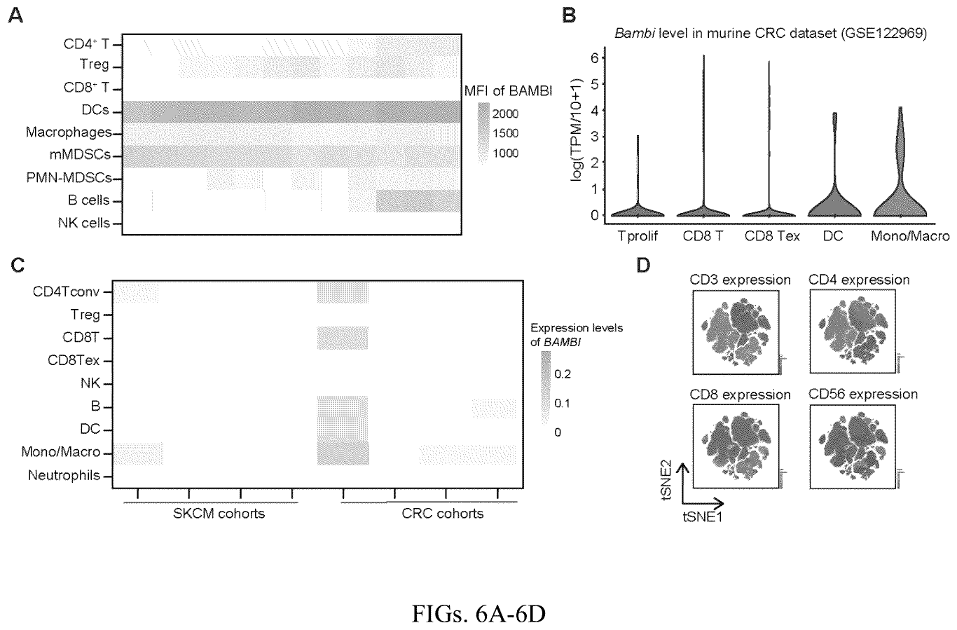

Next, the inventors used two murine cancer models to exclude the role of tumor cell-intrinsic BAMBI in antitumor activity. Overexpression of BAMBI did not impact the tumor growth of both MC38 colon cancer and B16 melanoma (FIGS. 5D-5F). The inventors speculated that BAMBI may mainly function in immune cells. To verify this, the inventors measured the expression level of BAMBI in different immune cells in MC38 tumors by flow cytometry analysis and found that BAMBI is predominantly expressed on myeloid-associated cells, including monocytes, macrophages and dendritic cells (FIG. 6A). Such a scenario is consistent with the analysis using a public single-cell RNA-seq (scRNA-seq) data in mice with colorectal adenocarcinoma (FIG. 6B). To dissect the main source of BAMBI in humans, the inventors analyzed public scRNA-seq data from the Tumor Immune Single-cell Hub (TISCH) and observed the relatively high expression of BAMBI in monocytes/macrophages in patients with melanoma or colorectal cancer (FIG. 6C). Furthermore, the inventors measured the protein levels of BAMBI in PBMC from cancer patients enrolled in a clinical trial at the institution (the COSINR study, NCT03223155; 26) and found monocytic MDSC (mMDSC) made up a sizable portion of BAMBI-positive cells (FIGS. 1A-1B and FIG. 6D).

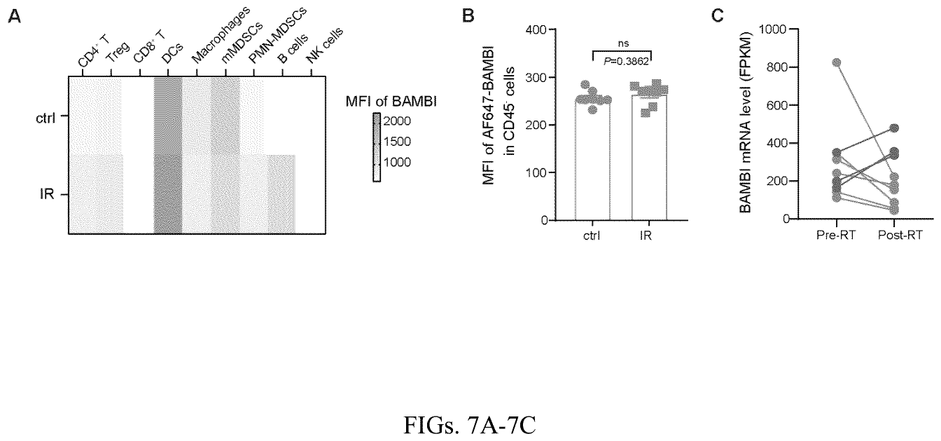

Considering the dominated role of myeloid cells in radioresistance (4, 24, 27), the inventors sought to investigate the effect of IR on BAMBI expression in myeloid cells compartments. The scRNA-seq data revealed that the mRNA level of Bambi was significantly decreased in the “monocyte_Ly6c2” population after IR (P=0.0105; FIG. 1C). The inventors also observed a significant reduction in Bambi among the “Macrophage_Mt-Co1” subset (P=0.0088; FIG. 1C). It is worth mentioning that “monocyte_Ly6c2”, identified as an mMDSC subset, exhibited the greatest proportionate increase among cell populations after IR, while a 2-fold decrease of “Macrophage_Mt-Co1” populations was observed following IR, as mentioned in a previous study (24). The inventors confirmed these findings by flow cytometry, demonstrating that BAMBI decreased in tumor infiltrating mMDSCs (FIGS. 1D-1E), but remained unchanged in other immune cells or CD45⁻ cells including tumor cells (FIGS. 7A-7B). In addition, the bulk RNA-seq and qPCR analysis confirmed the decreased mRNA expression of Bambi following IR (FIG. 1F-1G). In an effort to translate this finding to humans, the inventors calculated the mRNA levels of BAMBI in biopsies from cancer patients (the trial: NCT03223155; 26), patients received stereotactic body radiotherapy (SBRT) and ipilimumab plus nivolumab (ipi/nivo) immunotherapy. BAMBI was modestly decreased post-RT vs. pre-RT in six out of nine patients with lung/live metastasis (FIG. 7C). The inventors also evaluated the levels of BAMBI in PBMCs and observed a mild decrease of BAMBI in mMDSCs, not in other immune cells, from PBMCs following RT compared with matched pre-RT levels (FIG. 1H). The finding demonstrate that IR specifically reduces BAMBI expression in MDSCs in both patient and murine cancers.

### Example 2: IR Regulates BAMBI Expression in an m⁶A-YTHDF2-Dependent Manner

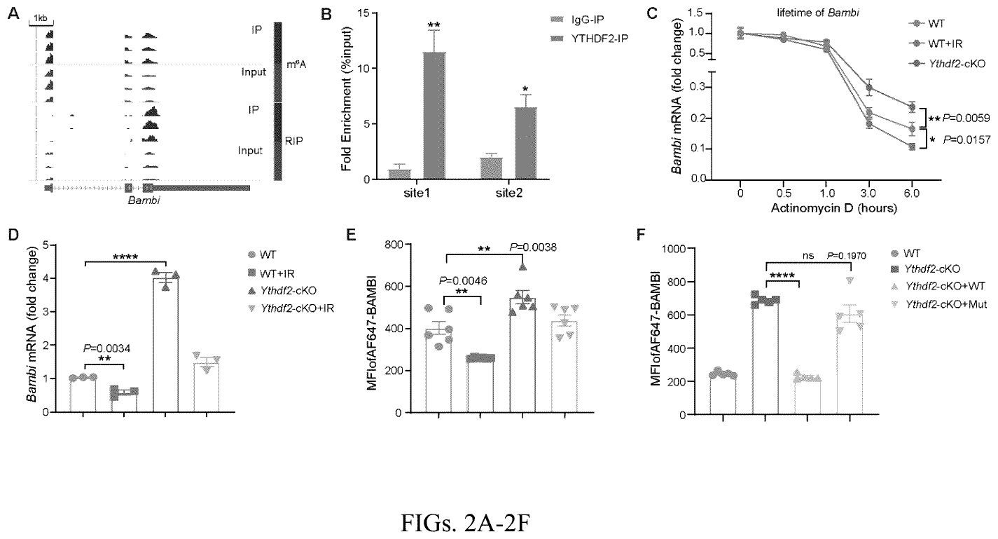

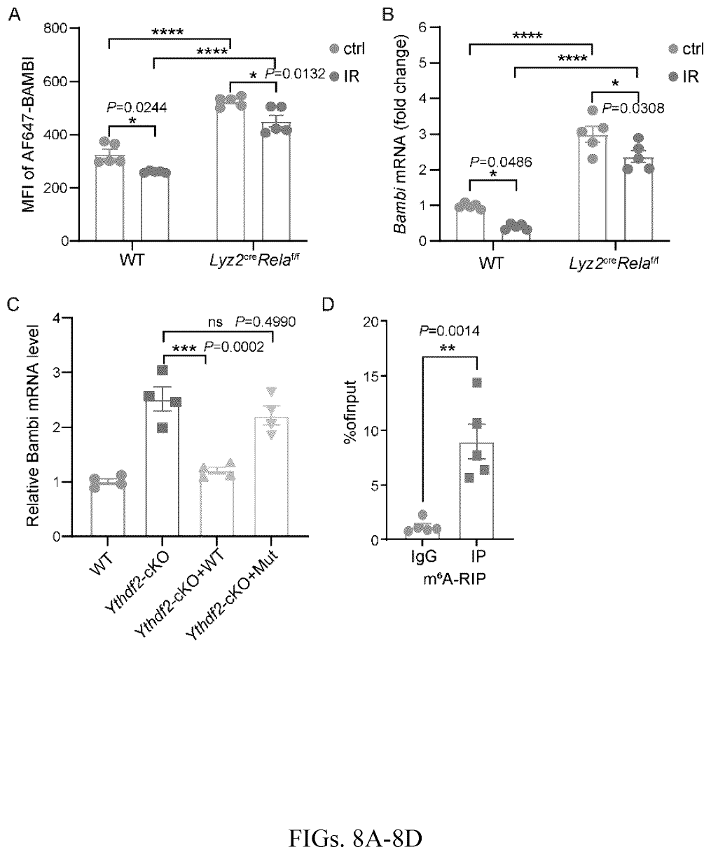

The inventors sought to ascertain the potential molecular mechanisms involved in radiation reduction of BAMBI in MDSCs. Considering that YTHDF2, an m⁶A reader protein, was dramatically induced by IR in myeloid cells (24), the inventors reanalyzed a prior dataset of mRNA-seq, N⁶-methyladenosine-sequencing RNA immunoprecipitation followed by high-throughput sequencing (MeRIP-seq) and RNA immunoprecipitation sequencing (RIP-seq) data of tumor-infiltrating CD11b⁺ myeloid cells to map direct target transcripts bound by YTHDF2. The results were highly reproducible among three biological replicates. The Integrative Genomics Viewer indicated a good fit between the m⁶A peaks and YTHDF2-binding peaks in the 3′UTR of Bambi (FIG. 2A). RIP-qPCR analyses with anti-YTHDF2 indicated that two sites were enriched in YTHDF2 binding in MDSCs compared to that with IgG control (FIG. 2B). Moreover, the transcript half-time of Bambi mRNA was significantly decreased in “WT+IR” MDSCs and increased in Ythdf2-cKO MDSCs (generated from Lyz2^{cre}Ythdf2^{f/f} conditional knockout mice) compared to WT-MDSCs (FIG. 2C), suggesting that IR promotes Bambi mRNA degradation via YTHDF2. The inventors further confirmed the BAMBI reduction in tumor-infiltrating MDSCs from “WT+IR” versus WT mice and the BAMBI induction in MDSCs from “Ythdf2-cKO” versus WT mice at both mRNA and protein levels (FIGS. 2D-2E). Since IR can induce YTHDF2 via NF-KB/RELA signaling in MDSCs (24), the inventors tested whether RELA is involved in BAMBI regulation. The inventors measured BAMBI expression in tumor-infiltrating MDSCs from Lyz2^{cre}Rela^{f/f} mice and found Rela knockout can increase BAMBI expression and rescue the reduction of BAMBI (FIG. 8A). These results highlight an IR-RELA-YTHDF2 axis that downregulates BAMBI in MDSC after IR.

To further demonstrate whether the aforementioned phenotype requires the m⁶A binding capacity of YTHDF2, the inventors force expressed YTHDF2 (Ythdf2-WT) and m⁶A-binding-site-mutated YTHDF2 (Ythdf2-Mut) in Ythdf2-deficient BM-MDSCs (CD45.2) and adoptively transferred these cells into MC38 tumor-bearing CD45.1 mice followed by IR treatment (20 Gy, one dose). Three days post-IR, the BAMBI level in newly infiltrated CD45.2-MDSCs in tumors was analyzed. Compared to the transferred WT-MDSCs, Ythdf2-cKO-MDSCs elicited significantly higher BAMBI levels (FIG. 2F and FIG. 8B). Overexpression of YTHDF2-WT in Ythdf2-cKO-MDSCs down-regulated BAMBI expression to the level in WT-MDSCs, whereas YTHDF2-Mut overexpression did not change BAMBI expression levels, which was maintained at a similar level in Ythdf2-cKO-MDSCs (FIG. 2F and FIG. 8B). Taken together, these observations indicate that an IR-RELA-YTHDF2 axis limits BAMBI production in MDSC in an m⁶A-dependent manner.

### Example 3: BAMBI Suppresses MDSC Migration and Suppressive Function Via Inhibiting TGF-β Signaling

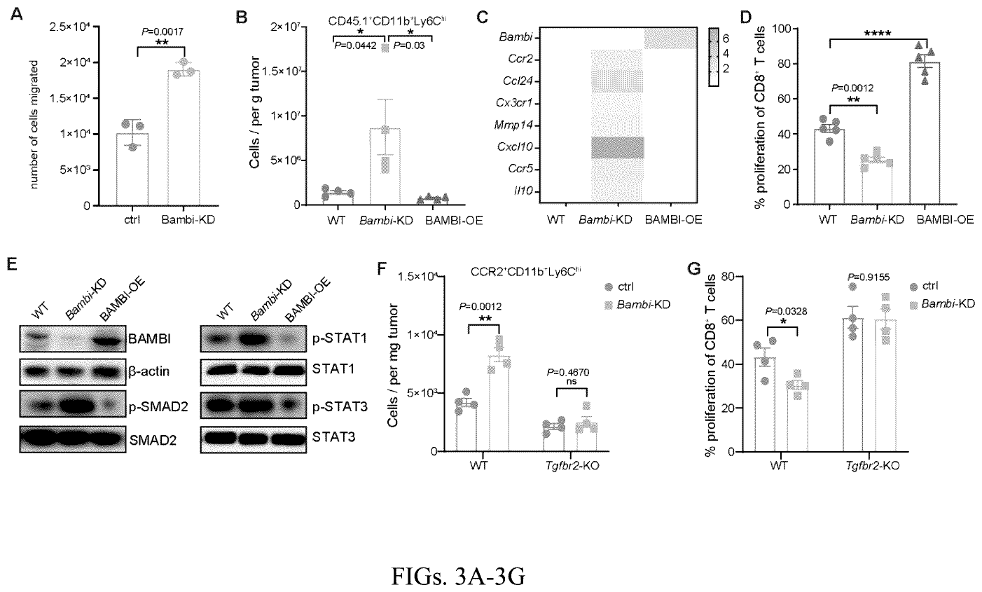

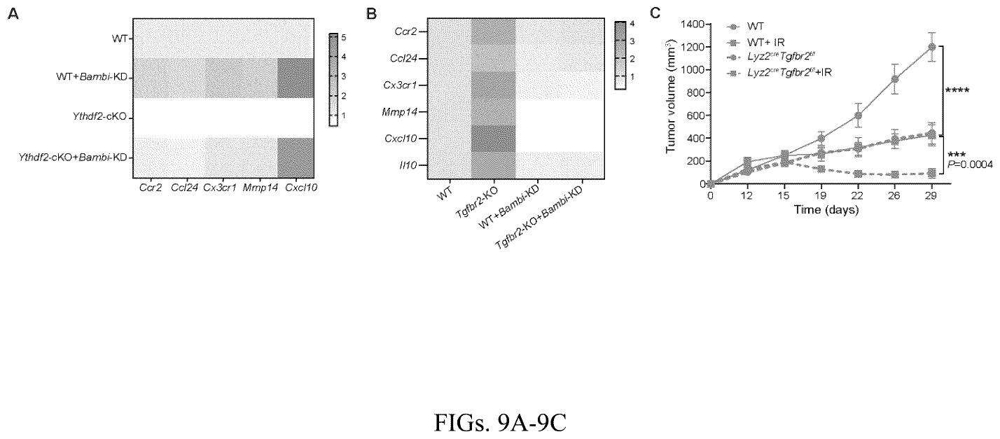

To investigate the functional role of BAMBI in MDSCs, the inventors generated Bambi-knockdown (Bambi-KD) bone marrow-derived MDSCs using siRNA and measured their migration activity. Transwell assay showed that Bambi-KD MDSCs elicited a significantly higher migration capacities than WT MDSC (FIG. 3A, P=0.0017). Subsequently, the inventors adoptively transferred Bambi-KD or BAMBI-overexpression MDSCs (CD45.1) into MC38 tumor-bearing Ccr2-knockout mice (CD45.2), in which radiation-induced MDSC infiltration is absent (4). Flow cytometry analysis revealed a significant increase of infiltrated MDSCs (CD45.1⁺CD11b⁺Ly6C^{hi}) in mice transferred with Bambi-KD MDSCs, while similar increase was not detected in mice transferred with BAMBI-overexpressing MDSCs upon radiation treatment (FIG. 3B). To examine any changes in underlying migration-associated genes, the inventors performed a series of qPCR analysis and found that the mRNA levels of Ccr2, Ccl24, Cx3crl, Ccr5, Cxcl10 and Mmp14 were significantly increased in Bambi-KD MDSC, and decreased in BAMBI-overexpressing MDSC (FIG. 3C). In addition, these genes were transcriptional regulated by the YTHDF2-BAMBI axis, as evidenced by rescued mRNA expression in Bambi-KD-Ythdf2-cKO MDSCs compared to those in Ythdf2-cKO MDSCs (FIG. 9A). These results demonstrate that YTHDF2 regulates BAMBI inhibition of MDSC migration both in vitro and in vivo.

Noteworthy, the inventors observed that Bambi knockdown led to a significant increase in Il10, whereas the overexpression of BAMBI decreased Il10 at mRNA level (FIG. 3C). Importantly, this was not observed for other immune suppression mediators (iNos2, Ros, Arg1, and Tgfb1), suggesting that BAMBI was not involved in their regulation. To further confirm the role of BAMBI in MDSC suppressive function, the inventors co-cultured Bambi-KD MDSCs with naïve CD8⁺ T cells and detected a reduction in T cell proliferation (FIG. 3D). The opposite result was observed when co-culturing BAMBI-overexpression MDSCs and CD8⁺ T cells (FIG. 3D). Collectively, the results demonstrate that IR decreases BAMBI to enhance MDSC migration and suppressive function, thereby contributing to radioresistance.

It is well known that TGF-β signaling facilitates MDSC migration, suppressive function, and the related chemokine/cytokine regulation (15, 17). It is thus conceivable that the functional role of BAMBI in MDSC requires TGF-β signaling. To pursuing this concept, the inventors first verified whether BAMBI impairs the TGF-β signaling in MDSCs. The inventors performed western blot to detect the level of phosphorylated SMAD2, a central downstream factor of TGF-β signaling, in WT or Bambi-KD MDSCs co-cultured with recombinant TGF-β. SMAD2 phosphorylation was increased in Bambi-KD MDSC and decreased in BAMBI-overexpressing MDSC compared to WT MDSC (FIG. 3E). TGF-β can directly activate the JAK1-STAT1/3 axis in a non-SMAD manner (28, 29). In concert with this, the western blot assay revealed the consistent changes in both STAT1 and STAT3 phosphorylation in Bambi-KD MDSC and BAMBI-overexpression MDSC (FIG. 3E). These findings indicate that BAMBI suppresses TGF-β signaling in MDSC.

To further confirm the involvement of TGF-β signaling in Bambi-KD-induced MDSC infiltration, the inventors generated Bambi-KD-Tgfbr2-KO MDSCs using bone marrow cells from Lyz^{Cre+}Tgfbr2^{fl/fl} conditional knockout mice (hereafter Tgfbr2-cKO) and used these constructs for the aforementioned transfer experiment. As expected, three days after IR, the level of infiltrating MDSC (Ccr2⁺CD11b⁺Ly6C^{hi}) was restored to a level similar to Tgfbr2-KO-MDSCs transfer (P=0.4670; FIG. 3F). Bambi-KD-Tgfbr2-KO MDSC also exhibited the comparable suppressive function with Tgfbr2-KO-MDSC (FIG. 3G). Functionally, the inventors observed consistent changes in the expression of genes associated with migration and suppressive function of MDSC, including Ccr2, Ccl24, Cx3crl, Mmp14, Cxcl10 and Il10 (FIG. 9B). To test if TGF-β in myeloid cells alters the response to radiotherapy, the inventors employed Tgfbr2-cKO and Tgfbr2^{fl/fl} (hereafter WT) for tumor growth experiments. In the syngeneic murine colon carcinoma (MC38) model, although primary tumor growth in Tgfbr2-cKO mice was slower than that in WT mice, local IR resulted in a more pronounced inhibition of tumor growth in Tgfbr2-cKO mice (FIG. 9C). Taken together, these data demonstrate that IR-deceased BAMBI suppresses TGF-β-mediated MDSC immune suppression.

### Example 4: Enhancing BAMBI Expression Improves the Responses to Radiotherapy

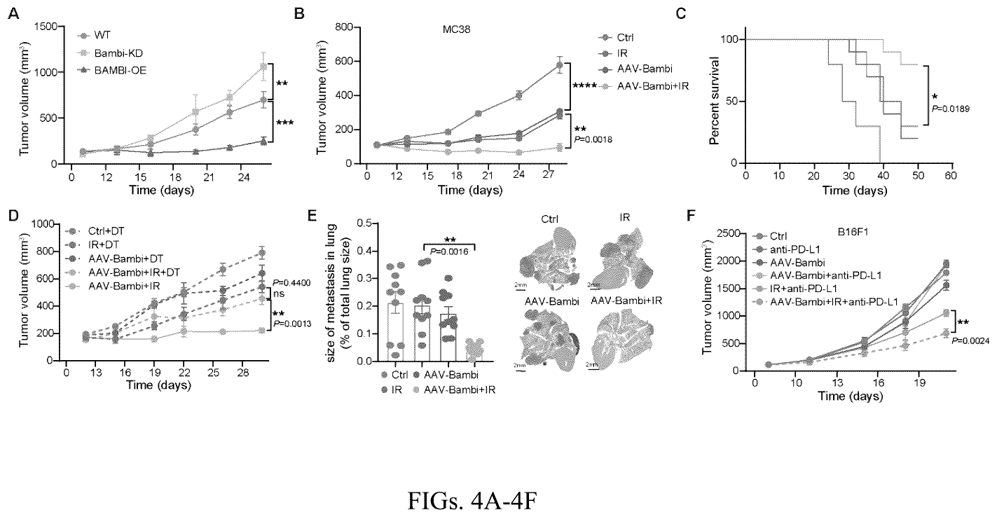

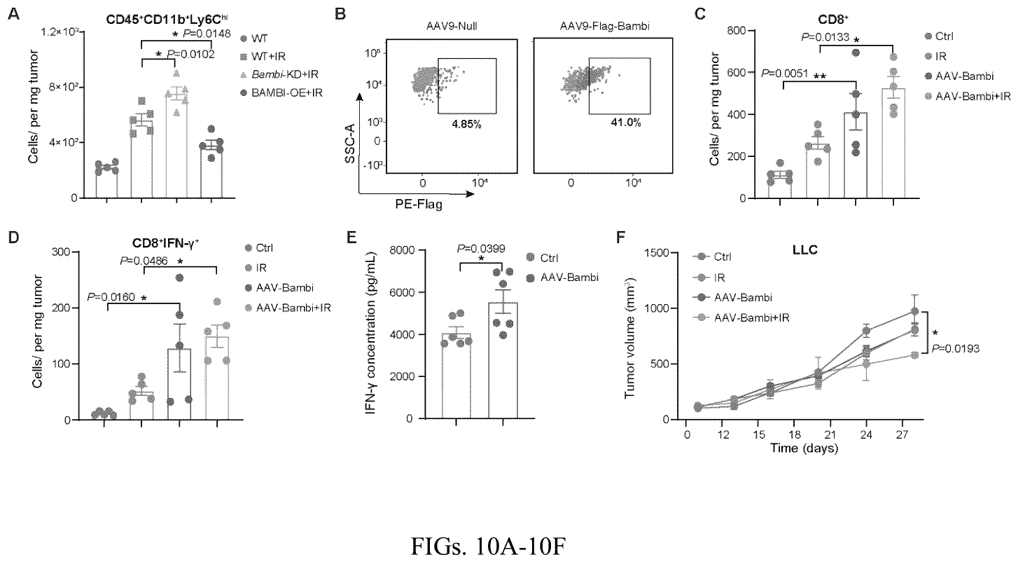

To determine the role of myeloid-specific BAMBI in controlling tumor growth, the inventors employed the bone marrow chimeric mice with CD45.1-bone marrow cells (BMCs) from CD11b-DTR mice for the following adaptive transfer experiments. Upon administration of diphtheria toxin, WT myeloid cells expressing CD11b-DTR are selectively eliminated, leaving the remaining modified CD11b⁺ cells. The inventors found that, following IR, MC38 tumors in mice transferred with Bambi-KD BMCs grew significantly faster, while MC38 tumors in mice transferred with BAMBI-overexpression BMCs grew remarkably slower compared to those transferred with WT BMCs (FIG. 4A). Consistently, the inventors observed a lower level of tumor-infiltrating MDSC in mice transferred with BAMBI-overexpression BMC with IR, and higher levels in those transferred Bambi-KD BMC (FIG. 10A). These results suggest that increasing BAMBI expression in myeloid cells can improve the response to radiotherapy.

To demonstrate that enhanced efficacy of radiation by BAMBI-overexpression can be translated into a clinically relevant strategy, the inventors generated an adeno-associated viral (AAV) vector containing BAMBI driven by a CMV promoter for in situ Bambi delivery. The inventors used an AAV9 capsid (hereafter AAV-Bambi), which is efficient and increasingly being utilized in clinical trials (30). AAV-mediated delivery of Bambi led to elevated expression of BAMBI in tumor-infiltrated MDSCs, as measured by flow cytometry 24 hours after i.t. injection (2×10¹⁰ v.p./dose) (FIG. 10B). To investigate its antitumor efficacy, the inventors performed in vivo studies in mice bearing MC38 tumors treated with AAV-Bambi (i.t. of 2×10¹⁰ v.p., twice weekly for two weeks). Although AAV-Bambi monotherapy did suppress tumor growth, the combination of AAV-Bambi and IR (20 Gy, one dose) caused a more pronounced suppressive effect on tumor growth compared to mice treated with IR alone, assessed in terms of both tumor size (P=0.0018) and animal survival (P=0.0189) (FIGS. 4B-4C).

The inventors observed that both tumor-infiltrating total CD8⁺ T cells and cytotoxic CD8⁺ T cells (IFNγ⁺CD8⁺) were significantly increased in “AAV-Bambi+IR” treated mice compared to IR alone (FIGS. 10C-10D). To investigate whether MDSCs are involved in the enhanced T cells function, the inventors tested the suppressive function of tumor-infiltrating MDSCs isolated from AAV-Bambi treated mice and observed an attenuated suppression function, as evidenced by a higher T cell IFN-γ production (FIG. 10E). Furthermore, the inventors measured the antitumor efficacy of AAV-Bambi in Lyz2^{cre}CSF1R-DTR mice, whose MDSCs were depleted after diphtheria toxin (DT) treatment. As expected, neither AAV-Bambi alone nor “AAV-Bambi+IR” lost the tumor inhibition ability (FIG. 4D), indicating that AAV-Bambi impairs MDSC suppressive function and thereby leads to the enhanced antitumor effect.

Intriguingly, in the LLC spontaneous lung metastasis model, a reduced metastatic burden (size of the lung metastasis) was observed in lungs of mice that received “AAV-Bambi+IR” treatment compared with mice that received IR alone (P=0.0016; FIG. 4E). Similarly, the inventors observed primary tumor control in “AAV-Bambi+IR” treated mice compared with control (P=0.0193; FIG. 10F). These data indicate that AAV-Bambi treatment enhanced the efficacy of radiotherapy through an increase in both local and distal metastasis control. The inventors also investigated whether AAV-Bambi could further increase the efficacy of IR and anti-PD-L1 treatment using a B16-melanoma model, which is resistant to ICB therapy. Compared with “IR+anti-PD-L1” treatment, triple combination treatment with AAV-Bambi, IR and anti-PD-L1 resulted in significantly slower tumor growth (FIG. 4F). The results demonstrate that AAV-Bambi can augment the response to radiotherapy and radio-immunotherapy combinations.

### Example 5: Implications of Aspects Herein

The inventors report that IR decreases BAMBI expression and in turn activates TGF-β signaling in MDSCs, resulting in a positive immune suppression feedback loop. BAMBI alters MDSC migration and suppressive function after IR. The study described herein delineates a previously unknown epitranscriptional-mechanism of BAMBI regulation whereby YTHDF2 directly binds to and triggers degradation of Bambi transcript in MDSCs. AAV delivery of Bambi into tumors not only improves local tumor control but also suppresses distant metastasis. The IR-YTHDF2-BAMBI-TGF-β circuit in MDSCs represents a previously unrecognized mechanism of extrinsic radioresistance.

Through single cell RNA-sequencing and flow cytometry analysis, the inventors are able to describe the distribution of BAMBI among immune cells in cancer patient PMBCs and murine colon (MC38) tumors in the context of IR. Considering that BAMBI is expressed at various levels in immune cells, the inventors speculate that BAMBI may affect their function and thereby impact host tumor immune response. Few studies have focused on the role of BAMBI in immune cells. For instance, the Merino group demonstrated that BAMBI modulates the differentiation of CD4⁺ cells during autoimmune arthritis (31). The functional role of BAMBI needs to be further explored in other immune populations, especially in the setting of cancer.

The findings that TGF-β signaling is required for MDSC migration and suppressive function after IR support previous observations that myeloid-specific TGF-β signaling is a critical mediator of tumor progression (16, 17). The inventors demonstrated that TGF-β signaling is under control of an YTHDF2-BAMBI axis in MDSC, providing the solid link with m⁶A modification and TGF-β signaling at the receptor level. The inventors demonstrated that NF-κB/RELA plays a central role in YTHDF2-mediated BAMBI expression in MDSC. Of note, the inventors cannot rule out the possibility that other NF-κB subunits might be involved in the regulation of BAMBI. For example, P50 could recruit HDACI to suppress Bambi transcription in HSCs (32). The inventors hypothesize that the IR-NF-κB axis contributes to MDSC migration and suppressive function via distinct mechanisms in the context of distinct conditions, resulting in an intricate network of NF-κB signaling in MDSC. These insights require detailed future mechanistic investigation.

Enhancing BAMBI in myeloid cells not only boosts the local anti-tumor effects of radiation, but also suppresses distant metastasis, amplifying the clinical importance of the findings. There are numerous anti-cancer pharmacological interventions that target specific mediators of TGF-β signaling pathway or TGF-β activators that have been tested in human clinical trials (33). However, adverse effects have presented a major challenge to the implementation of anti-TGF-β therapies, likely due to the dual nature of TGF-β signaling which can both function as a tumor suppressor in carcinoma cells and promote tumor progression in other contexts especially in immune cells (34, 35). The inventors demonstrated that BAMBI is mainly expressed in myeloid cells, especially MDSC, and therefore may be a better target (elevating myeloid cell-specific BAMBI) for inhibiting TGF-β signaling than the systemic TGF-β blockade.

### Example 6: Materials and Methods for Certain Aspects

### Cells and Reagents

MC38 and B16F1 were purchased from ATCC and were maintained according to the method of characterization used by ATCC. LLC cells were obtained from American Type Culture Collection (CRL-1642). Depleting or blocking antibodies against PD-L1 (BioXCell Cat #BE0101) were purchased from BioXCell. AAV-Bambi was produced by Vector Biolabs (QB59234).

### Mice

All mice were housed and used according to the animal experimental guidelines set by the Institute of Animal Care and Use Committee of The University of Chicago. All animals were maintained in pathogen-free conditions and cared for in accordance with the International Association for Assessment and Accreditation of Laboratory Animal Care policies and certification. Ythdf2^{flox/flox} mice were generated using CRISPR-Cas9 technology as described (36). Lyz^{Cre} mice, CD45.1 mice, Rela^{flox/flox} mice, Ccr2^{-/-} mice, and Tgfbr2^{flox/flox} mice were purchased from The Jackson Laboratory. The inventors used female mice with 6-8 weeks of age for all experiments.

### Patient Sample

Patient samples (PBMCs) were obtained from patients treated at the University of Chicago enrolled in the trial and NCT03223155 (26).

### Tumor Growth and Treatment

1×10⁶ MC38, LLC, or B16-F1 tumor cells were subcutaneously (s.c.) injected in the right flank of mice. Mice were pooled and randomly divided into different groups when the tumor reached a volume of approximately 100 mm³ (L×W×H×0.5), but not blinded. The mice were treated with 20 Gy of tumor-localized radiation (one dose) or sham treatment. For anti-PD-L1 treatment experiments, 200 μg of the anti-PD-L1 antibody were injected intraperitoneally twice each week for a total of three times. For AAV-Bambi delivery experiments, the tumor-bearing mice were injected intratumorally with 2×10¹⁰ v.p. of either AAV-Bambi or AAV-null, twice each week for a total of four times. For adoptive transfer, 5×10⁶ MDSCs were injected intravenously (i.v.) into recipient mice (chimeric mice); on the same day, mice were received 20 Gy of tumor-localized radiation (one dose). Tumors were measured twice one week for 3-4 weeks. Animals were euthanized when the tumor volume reached 2,000 mm³ or the diameter of tumor reached 1.5 cm (according to the IACUC protocol).

### Overexpression of BAMBI in Tumor Cells

The mouse Bambi cDNA was cloned into the lentiviral expression vector pLJM1 to construct the vector pLJM1-BAMBI and packaged by co-transfection of 293-T cells with two lentiviral helper plasmids-pMD2.G and psPAX2. Virus-containing conditioned medium was harvested 48 h to 72 h after transfection, filtered, and used to infect MC38 or B16F1 cells in the presence of 8 μg/mL polybrene. Infected cells were selected with 2-10 μg/mL puromycin. The BAMBI-overexpressing cells were confirmed by qPCR analysis.

### Flow Cytometry

For flow cytometric analysis, tumors were collected from mice. The collected tumors tissues were cut into small pieces and were digested with 1 mg/ml collagenase type I (Fisher) and 200 μg/ml DNasel (Sigma-Aldrich) at 37° C. for 60 min to generate the single-cell suspensions. Samples were then filtered through a 70 μm cell strainer and washed twice with staining buffer (PBS supplemented with 2% FBS and 0.5 mM EDTA). The cells were re-suspended in staining buffer and were blocked with anti-FcR (2.4G2, BioXcell). Subsequently, the cells were stained with 100˜200-fold diluted fluorescence-labeled antibodies for 30 min at 4° C. in the dark and then detected by flow cytometry with a BD Fortessa (BD). For BAMBI staining, the primary anti-BAMBI antibody (Thermo Fisher Scientific Cat #PA5-38027, RRID: AB_2554631) was first conjugated with secondary antibody with Alexa Fluor® 488 Conjugation Kit (Fast)—Lightning-Link® (Abcam) and then was used for flow staining. Analysis of flow cytometry data was performed using FlowJo V10.

Multicolor spectral flow cytometric analysis was performed on patient PBMCs using a 28-color antibody panel on Cytek Aurora (Cytek Biosciences, Fremont, CA). 5×10⁴ live CD45⁺ cells from each sample were concatenated and used for further downstream analysis. High-dimensional data was visualized using t-distributed stochastic neighbor embedding (t-SNE) in FlowJo 10.8.1 (BD, Franklin Lakes, NJ). Phenograph v2.4 (37) and FlowSOM v3.0.18 (38) were used for unsupervised nearest-neighbor clustering based on phenotypic similarities. FlowJo plugin Cluster explorer v.1.7.4 (BD, Franklin Lakes, NJ) was used for data visualization.

### Bone Marrow-Derived MDSCs Induction and Isolation

Bone marrow was obtained from wild type, Ythdf2^{f/f} or Lyz^{cre}Ythdf2^{f/f} mice and was used to prepare single cell suspension (fresh bone marrow cells). The cells were cultured in RPIM-1640 medium containing 10% FBS and 20 ng/ml Recombinant Mouse GM-CSF carrier-free (BioLegend). Fresh medium supplemented with GM-CSF was added on day 3. On day 4, the bone marrow-derived MDSCs (BM-MDSCs) were obtained from fresh bone marrow cells followed with MDSCs isolation using EasySep Monocytes Selection kits (STEMCELL Technologies).

### MDSC Suppression Assay

Murine MDSCs purified from tumors or bone marrow derived MDSCs were performed for the suppression assay. CD8⁺ T cells isolated from the spleens of naive mice by using EasySep™ Mouse CD8⁺ T Cell Isolation Kit (STEMCELL) according to manufacturer's instructions and then stained with CellTrace CFSE (Invitrogen). The CD8⁺ T cells were cultured with anti-CD3/anti-CD28 beads and were co-cultured with MDSCs at a ratio of 4:1. The CD8⁺ T cells proliferation was analyzed by flow cytometry or the IFN-γ concentration was measured by CBA flex set (BD).

### Knockdown of Bambi in BM-MDSCs

SiRNA targeting mouse Bambi was transfected into bone marrow derived MDSCs by TransIT-TKO® Transfection Reagent (Mirus) according to manufacturer's protocol. The sequence of siRNA is: (sense) 5′-CCA-GAC-UUC-UCG-AUC-CUC-Att-3′. One-two days after the transfection, the cells were collected. The knockdown efficiency was detected by qPCR.

### Transwell Migration Assay

The inventors used 24-well transwell plates with 8 μm inserts in polyethylene terephthalate track-etched membranes (Corning). The purified MDSCs from tumors or bone marrow derived cells (1.5×10⁶ cells/insert) in serum-free medium were added into the upper compartment of the chamber. The inserts were placed in plates with complete DMEM medium. After incubating overnight, insert membranes were washed with PBS, fixed with 70% methanol for 10 min, and stained with 0.05% crystal violet to detect the migrated cells. An inverted microscope was used for counting.

### RNA Stability Assay

MDSCs were sorted from spleen in Ythdf2^{f/f} and Lyz^{cre}Ythdf2^{f/f} mice and were seeded in 24-well plates at 50% confluency. For IR treatment, MDSCs were treated with IR (4 Gy) and cultured for 6 h. 5 μg/mL of Actinomycin D (Sigma-Aldrich) was added. After 0, 0.5, 1, 3, and 6 hours of incubation, cells were collected. The total RNA was purified by RNeasy kit with an additional DNase-I digestion step on the column. RNA quantities were determined using qPCR.

### Forced Expression of m6A-Binding-Site-Mutated YTHDF2 in MDSCs

The cloned Ythdf2 cDNA with K416A, R527A, W432A, and W486A mutation, which has been proved to significantly decrease the m⁶A binding affinity (39), were synthesized and cloned into the lentiviral expression vector pLVX-ZsGreen-N1 to generate pLVX-ZsGreen-N1-Ythdf2-Mut (GenScript). The constructed vectors were packaged by co-transfection of 293X cells with two lentiviral helper plasmids pVSVG and pVPR. Virus-containing conditioned medium was harvested 48 h after transfection, filtered, and used to infect BM-MDSCs in the presence of 8 μg/mL polybrene. Infected cells were selected with 2 μg/mL puromycin.

### Generation of Bone Marrow Chimeras

WT mice were irradiated with a single dose of 8 Gy. The irradiated mice were adoptively transferred (i.v.) with 5×10⁶ WT BMCs from CD11b-DTR mice. The mice were treated with Neomycin (0.5 mg/mL) diluted in drinking water for 4 weeks after reconstitution and then were used for the adaptive transfer experiments after eight weeks.

### RIP-qPCR Analysis

RIP for YTHDF2 was performed using 10-20 μg anti-YTHDF2 rabbit polyclonal antibody (Aviva systems biology). After IP, RNA was isolated from Input and IP fractions using phenol/chloroform extraction. cDNA was prepared with the Applied Biosystems™ High-Capacity cDNA Reverse Transcription Kit (Thermo). SYBR-green-based qPCR was performed using QuantiStudio3 (ABI).

### Immunoblot Analysis

Whole-cell protein was extracted with RIPA buffer supplemented with Protease/Phosphatase Inhibitor Cocktail (100×) (Thermo Scientific). Protein concentrations were measured by BCA Protein Assay Kit (Pierce). Equal amounts of protein were separated by SDS-PAGE and transferred to PVDF membrane (Invitrogen). Membranes were blocked in 1% BSA or 5% milk for 1 h and incubated with the relevant primary antibodies at 4° C. overnight. Next, the membrane was washed with Tris-buffered saline with 0.1% Tween20 (TBST) and incubated with appropriate secondary HRP antibodies for another 1 h. The membrane was then washed with TBST and ECL was applied for film development. The amount of loaded protein was normalized to β-actin.

### Single Cell RNA-seq (scRNA) Analysis

CD45⁺ single-cell suspensions were obtained from four pooled MC38 tumors in WT mice with or without IR (20 Gy) four days after IR. Raw scRNA-seq data were processed into FASTQ files using “cellranger mkfastq” function of 10X Genomics Cell Ranger (v6.0.1), and the reads were then aligned to the mouse reference genome mm10 (40). The unique molecular identifier (UMI) for each gene in each cell was counted with “cellranger count” function (40). Then low-quality cells were discarded if containing (1) less than 200 expressed genes; or (2) more than 25% percentage of mitochondrial genes. The doublets were removed by DoubletFinder (v2.0.3) assuming 6% doublet formation rate (41). The processed whole gene expression matrix was then fed to Seurat (v4.0.6) for downstream analysis (42).

Further, UMI count matrix from Ctrl and IR samples were integrated by Seurat: : IntegrateData method after normalization with ‘SCT’ method using 3,000 highly variable feature genes (42). Clustering analysis were performed using the first 40 principal components for constructing the shared nearest neighbor (SNN) graph with resolution as 0.7. The marker genes were called by Seurat::FindAllMarkers (pct.1>=0.6 & Log 2FC>=1). Then, identified clusters were annotate by scClassify (v1.2.0) (43) based on cell types of hierarchies constructed from the reference dataset (E-MTAB-8832, CD45⁺ immune cells sorted from MC38 tumor bearing C57BL/6 mice) (44) and labeled using the selected feature gene. The myeloid cells, which were gathered closely and annotated as macrophage, monocyte or DC2 cells, were extracted and were clustered into several sub-clusters. They were further annotated using the same methods as above. Heatmap of Bambi expression was plot based on the average normed reads count of this gene across cells from IR or Ctrl sample.

### AAV-Bambi Production

Mouse BAMBI cDNA open reading frame (accession No. NM_026505.2) was cloned into pcDNA3.1+/C-(K)-DYK vector and made available by Genscript (USA). The BAMBI-DYK was subsequently subcloned into a pAAV cis-plasmid driven by CMV, with AAV2 ITR to make AAV9 capsid by Vector BioLab (USA). The titer of the particles is 2.6×10¹³ GC/mL.

### Statistical Analysis

To estimate the statistical significance of differences between two groups, the inventors used an un-paired Student's t-tests to calculate two-tailed P values. One-way analysis of variance (ANOVA) or two-way ANOVA with multiple comparison test was performed when more than two groups were compared. Survival analysis was performed using Kaplan-Meier curves and evaluated with log-rank Mantel-Cox tests. Error bars indicate the standard error of the mean (SEM) unless otherwise noted. P values are labeled in the figures. P values were denoted as follows: *P<0.05, **P<0.01, ***P<0.001, ****P<0.0001. Statistical analyses were performed by using GraphPad Prism (version 9.0).

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Barker H E, et al. The tumour microenvironment after radiotherapy: mechanisms of resistance and recurrence. Nat Rev Cancer. 2015;15:409-425.
2. Weichselbaum R R, et al. Radiotherapy and immunotherapy: a beneficial liaison? Nat Rev Clin Oncol. 2017;14:365-379.
3. Formenti S C, Demaria S. Systemic effects of local radiotherapy. Lancet Oncol. 2009;10:718-26.
4. Liang H, et al. Host STING-dependent MDSC mobilization drives extrinsic radiation resistance. Nat Commun. 2017;8:1736.
5. Twyman-Saint Victor C, et al. Radiation and dual checkpoint blockade activate non-redundant immune mechanisms in cancer. Nature. 2015;520:373-377.
6. Vanpouille-Box C, et al. TGFβ is a master regulator of radiation therapy-induced antitumor immunity. Cancer Res. 2015;75:2232-42.
7. Chen H M, et al. Myeloid-derived suppressor cells as an immune parameter in patients with concurrent sunitinib and stereotactic body radiotherapy. Clin Cancer Res. 2015;21:4073-4085.
8. Gentles A J, et al. The prognostic landscape of genes and infiltrating immune cells across human cancers. Nat Med. 2015;21:938-945.
9. Parker K H, et al. Myeloid-derived suppressor cells: critical cells driving immune suppression in the tumor microenvironment. Adv Cancer Res. 2015;128:95-139.
10. Engblom C, et al. The role of myeloid cells in cancer therapies. Nat Rev Cancer. 2016;16: 447-462.
11. Kumar V, et al. The nature of myeloid-derived suppressor cells in the tumor microenvironment. Trends Immunol. 2016;37:208-220.
12. Smith A L, et al. Molecular pathways: targeting the TGF-beta pathway for cancer therapy. Clin Cancer Res. 2012;18:4514-4521.
13. Kim B G, et al. Novel therapies emerging in oncology to target the TGF-beta pathway. J Hematol Oncol. 2021;14:55.
14. Ciardiello D, et al. Clinical development of therapies targeting TGFβ: current knowledge and future perspectives. Ann Oncol. 2020;31:1336-1349.
15. Tzavlaki K, Moustakas A. TGF-β Signaling. Biomolecules. 2020;10:487.
16. Kim B G, et al. Smad4 signalling in T cells is required for suppression of gastrointestinal cancer. Nature. 2006;441:1015-9.
17. Geissmann F, et al. TGF-beta 1 prevents the noncognate maturation of human dendritic Langerhans cells. J Immunol. 1999;162:4567-75
18. Wahl S M, et al. Transforming growth factor type beta induces monocyte chemotaxis and growth factor production. Proc Natl Acad Sci USA. 1987;84:5788-92.
19. Batlle E, Massague J. Transforming growth factor-beta signaling in immunity and cancer. Immunity. 2019;50:924-940.
20. Novitskiy S V, et al. Deletion of TGF-beta signaling in myeloid cells enhances their anti-tumorigenic properties. J Leukoc Biol. 2012;92:641-651.
21. Pang Y L, et al. TGF-beta signaling in myeloid cells is required for tumor metastasis. Cancer Discov. 2013;3:936-951.
22. Legg K. Autoimmunity: A controlled performance by BAMBI. Nat Rev Rheumatol. 2016;12:72.
23. Onichtchouk D, et al. Silencing of TGF-beta signalling by the pseudoreceptor BAMBI. Nature. 1999;401:480-5.
24. Marwitz S, et al. Downregulation of the TGFβ pseudoreceptor BAMBI in non-small cell lung cancer enhances TGFβ signaling and invasion. Cancer Res. 2016;76:3785-801.
25. Sekiya T, et al. Transcriptional regulation of the TGF-beta pseudoreceptor BAMBI by TGF-beta signaling. Biochem Biophys Res Commun. 2004;320:680-684.
26. Fritzmann J, et al. A colorectal cancer expression profile that includes transforming growth factor beta inhibitor BAMBI predicts metastatic potential. Gastroenterology. 2009;137:165-75.
27. Khin S S, et al. BAMBI gene is epigenetically silenced in subset of high-grade bladder cancer. Int J Cancer. 2009;125:328-38.
28. Wang L, et al. YTHDF2 inhibition potentiates radiotherapy antitumor efficacy. Cancer Cell. 2023;41:1-15.
29. Kovács S A, Györffy B. Transcriptomic datasets of cancer patients treated with immune-checkpoint inhibitors: a systematic review. J Transl Med. 2022;20:249.
30. Li T, et al. TIMER2.0 for analysis of tumor-infiltrating immune cells. Nucleic Acids Res. 2020;48:W509-W514.
31. Bestvina C M, et al. A Phase 1 trial of concurrent or sequential ipilimumab, nivolumab, and stereotactic body radiotherapy in patients with stage IV NSCLC study. J Thorac Oncol. 2022;17:130-140.
32. Jiménez-Cortegana C, et al. Myeloid-derived suppressor cells and radiotherapy. Cancer Immunol Res. 2022;10:545-557.
33. Derynck R, Zhang Y E. Smad-dependent and Smad-independent pathways in TGF-beta family signalling. Nature. 2003;425:577-584.
34. Tang L Y, et al. Transforming growth factor-beta (TGF-beta) directly activates the JAK1-STAT3 axis to induce hepatic fibrosis in coordination with the SMAD pathway. J Biol Chem. 2017;292:4302-4312.
35. Wang D, et al. Adeno-associated virus vector as a platform for gene therapy delivery. Nat Rev Drug Discov. 2019;18:358-378.
36. Postigo J, et al. Bone Morphogenetic Protein and Activin Membrane-Bound Inhibitor, a transforming growth factor β rheostat that controls murine Treg Cell/Th17 cell differentiation and the development of autoimmune arthritis by reducing interleukin-2 signaling. Arthritis Rheumatol. 2016;68:1551-62.
37. Liu C, et al. Transcriptional repression of the transforming growth factor β (TGF-β) pseudoreceptor BMP and activin membrane-bound inhibitor (BAMBI) by nuclear factor κB (NF-κB) p50 enhances TGF-β signaling in hepatic stellate cells. J Biol Chem. 2014;289:7082-7091.
38. Liu S, et al. Targeting TGF-β signal transduction for cancer therapy. Signal Transduct Target Ther. 2021;6:8.
39. Bierie B, Moses H L. TGF-beta and cancer. Cytokine Growth Factor Rev. 2006;17:29-40.
40. Massague, J. TGF beta in cancer. Cell. 2008;134:215-230.
41. Li M, et al. Ythdf2-mediated m⁶A mRNA clearance modulates neural development in mice. Genome Biol. 2018;19:69.
42. Levine J H, et al. Data-driven phenotypic dissection of aml reveals progenitor-like cells that correlate with prognosis. Cell. 2015;162:184-97.
43. Van Gassen S, et al. FlowSOM: Using self-organizing maps for visualization and interpretation of cytometry data. Cytometry A. 2015;87:636-45.
44. Zhu T, et al. Crystal structure of the YTH domain of YTHDF2 reveals mechanism for recognition of N⁶-methyladenosine. Cell Res. 2014;24:1493-6.
45. Zheng G X, et al. Massively parallel digital transcriptional profiling of single cells. Nat Commun. 2017;8:14049.
46. McGinnis C S, et al. DoubletFinder: doublet detection in single-cell rna sequencing data using artificial nearest neighbors. Cell Syst. 2019;8:329-337.e4.
47. Stuart T, et al. Comprehensive integration of single-cell data. Cell. 2019;177:1888-1902.e21.
48. Lin Y, et al. scClassify: sample size estimation and multiscale classification of cells using single and multiple reference. Mol Syst Biol. 2020;16:e9389.
49. Zhang L, et al. Single-cell analyses inform mechanisms of myeloid-targeted therapies in colon cancer. Cell. 2020;181:442-459.e29.

1. A population of immune cells comprising an exogenous bone morphogenetic protein and activin membrane-bound inhibitor (BAMBI) gene product.

2. The population of immune cells of claim 1, wherein the population comprises myeloid-derived suppressor cells.

3. The population of immune cells of claim 1, wherein the population consists essentially of myeloid-derived suppressor cells.

4. The population of immune cells of claim 1, wherein the population consists of myeloid-derived suppressor cells.

5. The population of immune cells of claim 1, wherein the population comprises myeloid precursor cells.

6. The population of immune cells of claim 1, wherein the population consists essentially of myeloid precursor cells.

7. The population of immune cells of claim 1, wherein the population consists of myeloid precursor cells.

8. The population of immune cells of claim 1, wherein the exogenous BAMBI gene product is a BAMBI RNA.

9. The population of immune cells of claim 1, wherein the exogenous BAMBI gene product is a BAMBI protein.

10. The population of immune cells of claim 1, wherein the exogenous BAMBI gene product is introduced to the population of immune cells by a viral vector.

11. The population of immune cells of claim 10, wherein the viral vector comprises an adeno-associated virus, an adenovirus, a retrovirus, a lentivirus, a herpes simplex virus, or a combination thereof.

12. The population of immune cells of claim 10, wherein the viral vector comprises an adeno-associated virus.

13. The population of immune cells of claim 1, wherein the exogenous BAMBI gene product is introduced to the population of immune cells by a non-viral vector.

14. The population of immune cells of claim 13, wherein the non-viral vector comprises a lipid delivery system, a cationic polymer, a conjugate complex, or a combination thereof.

15. (canceled)

16. The population of immune cells of claim 1, wherein the exogenous BAMBI gene product induces an overexpression of BAMBI protein in the population of immune cells.

17. The population of immune cells of claim 16, wherein the overexpression is relative to a population of immune cells that do not contain the exogenous BAMBI gene product.

18. A pharmaceutical composition comprising the population of immune cells of claim 1.

19. The pharmaceutical composition of claim 18, wherein the population of immune cells comprises between 1×10³ to 9×10¹² cells.

20. A method of enhancing a patient's responsiveness to a cancer therapy, the method comprising increasing expression of bone morphogenetic protein and activin membrane-bound inhibitor (BAMBI) in a population of immune cells in the patient.

21.-32. (canceled)

33. The method of claim 20, wherein the increasing expression of BAMBI comprises administering a BAMBI overexpression construct to the patient.

34.-47. (canceled)

48. A method of treating cancer in a patient, the method comprising increasing expression of bone morphogenetic protein and activin membrane-bound inhibitor (BAMBI) in a population of immune cells.

49.-60. (canceled)

61. The method of claim 48, wherein the increasing expression of BAMBI comprises administering a BAMBI overexpression construct to the patient.

62.-72. (canceled)

73. A method of predicting responsiveness to a radiotherapy and/or immunotherapy in a patient, the method comprising measuring an amount of bone morphogenetic protein and activin membrane-bound inhibitor (BAMBI) in a population of immune cells, wherein the patient has received, will receive, and/or is indicated to receive the radiotherapy.

74.-83. (canceled)
